(19) United States
(12) Patent Application Publication
(10) Pub. No.: US 2025/0375369 A1
(43) Pub. Date: Dec. 11, 2025

(54) COMPOSITIONS COMPRISING SELF TANNING AGENTS AND CERTAIN SPHINGOLIPIDS AND/OR CERTAIN SPHINGOID BASES

(71) Applicant: Evonik Operations GmbH, Essen (DE)

(72) Inventors: Jennifer Schild, Solingen (DE); Xin Lu, Essen (DE); Benjamin Tuchscherer, Dortmund (DE); Ursula Maczkiewitz, Essen (DE); Sandra Nattland, Essen (DE); Marita Regina Baetsen, Issum (DE); Lisa Maus, Duisburg (DE); Monica Desiree van Logchem, PA Zevenbergen (NL)

(73) Assignee: Evonik Operations GmbH, Essen (DE)

(21) Appl. No.: 18/876,115

(22) PCT Filed: Jun. 19, 2023
(86) PCT No.: PCT/EP2023/066400

§ 371 Date: Dec. 17, 2024

(30) Foreign Application Priority Data
Jul. 1, 2022 (EP) 22182451.9

(51) Int. Cl. A61K 8/55; A61K 8/35; A61Q 19/04

(52) U.S. Cl. CPC A61K 8/553; A61K 8/35; A61Q 19/04

(57) ABSTRACT

A composition has at least one self tanning agent and certain sphingolipids and/or certain sphingoid bases. Cosmetic formulations having the composition with at least one self tanning agent, certain sphingolipids and/or certain sphingoid bases enhance a self tanning effect of a self tanning agent or find application in cosmetic products for tanning of a human skin.

### FIELD OF THE INVENTION

The invention relates to compositions comprising at least on self tanning agent and certain sphingolipids and/or certain sphingoid bases.

### PRIOR ART

Ceramides as such have been used within the cosmetic field for decades.

There are products out in the market containing Ceramide NP (Ceramide III, N-oleoyl-phytosphingosine) and self tanning agents.

However, so far, no special effect in the field of tanning skin has been attributed to the class of ceramides.

It is an object of the invention to provide substances, that enhance the self tanning effect in cosmetics.

### DESCRIPTION OF THE INVENTION

It was found that, surprisingly, that certain sphingolipids and certain sphingoid bases can boost the tanning effect of self tanning agents.

The present invention therefore provides a composition as described in claim 1.

The invention further provides new sphingolipids as described in claim 8.

One advantage of the present invention is that sphingolipids can improve the tanning effect of self-tanning agents without inducing commonly known uv-damage reactions which occur through sun-light exposure of the skin.

As additional benefit, the barrier is strengthened by sphingolipid application.

Another advantage of the present invention is that the addition of Sphingolipids to self-tanning agents will reduce the negative effects of those molecule class. DHA as an example is known to form Formaldehyde in the skin after application. By the addition of sphingolipids the concentration of self-tanning molecules like DHA can be significantly reduced and side-effects can be minimized.

A further advantage is that the skin tone will be more even by addition of sphingolipids and less yellow as it is known for classical self-tanning molecules.

In addition, the self-tanning effect will last longer. Sphingolipids are highly effective active ingredients which already work in low concentrations

Another advantage is a better formulation stability, because sphingolipids build lamellar phases which can help to stabilize classical self-tanning molecules.

Another advantage of the present invention is that sphingolipids, especially the structures described in claim 8, will strengthen, modulate and balance the skin microbiome which contributes to a strong skin-barrier and an overall healthy skin with prolonged tanning.

The present invention thus provides a composition comprising
A) at least on self tanning agent
B) at least one
sphingolipid of the general formula I

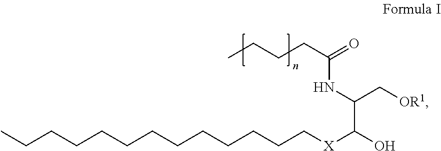

where
R¹ is H, phosphocholine, serine, ethanolamine or a sugar, preferably a sugar or H, more preferably H,
X is CH═CH, CH₂—CH₂ or CH₂—HCOH, preferably CH₂—HCOH, and
n is selected from an integer from 3 to 8, preferably from 6 and 7, most preferably 6, and/or at least one
sphingoid base selected from the group of N-acetylphytosphingosine, phytosphingosine, sphingosine and sphinganine.

The sphingolipid of the general formula I has a plurality of stereogenic centres, all of which are covered by the general formula I.

Unless stated otherwise, all percentages (%) given are percentages by mass.

Preferred compositions according to the instant invention are characterized, in that the at least on self tanning agent is selected from the group of dihydroxyacetone und erythrulose.

Preferred compositions according to the instant invention are characterized, in that the sphingolipid of the general formula I is chosen from those, where
R¹ is H,
X is CH₂—HCOH, and
n is selected from an integer from 6 and 7, most preferably 6.

Preferred compositions according to the instant invention are characterized, in that A) is comprised in an amount of 0.02% by weight to 1.50% by weight, preferably of 0.03% by weight to 1.00% by weight, more preferably of 0.05% by weight to 0.50% by weight, the percentages by weight being based on the total composition.

Preferred compositions according to the instant invention are characterized, in that B) is comprised in an amount of 0.5% by weight to 15.0% by weight, preferably of 1.0% by weight to 10.0% by weight, more preferably of 3.0% by weight to 6.0% by weight, the percentages by weight being based on the total composition.

The compositions according to the instant invention are preferably cosmetic formulations.

Cosmetic formulations containing self tanning agents are frequently used by people, who want to avoid direct exposure of their skin to UV light of the sun, but still want to benefit from the advantage of a tanned skin.

Therefore, preferred cosmetic formulations according to the instant invention comprise at least one UV light protection filter substance.

The UV light protection filters used may be for example organic substances capable of absorbing ultraviolet radiation and then re-emitting the absorbed energy in the form of longer-wave radiation, for example heat.

UVB filters may be oil-soluble or water-soluble. Examples of oil-soluble UVB light protection filters include:
3-benzylidenecamphor and derivatives thereof, for example 3-(4-methylbenzylidene) camphor, 4-aminobenzoic acid derivatives, for example 2-ethylhexyl 4-(dimethylamino)benzoate and amyl 4-(dimethylamino)benzoate,
esters of cinnamic acid, for example 2-ethylhexyl 4-methoxycinnamate, isopentyl 4-methoxycinnamate, 2-ethylhexyl 2-cyano-3-phenylcinnamate (Octocrylene), esters of salicylic acid, for example 2-ethylhexyl salicylate, 4-isopropylbenzyl salicylate, homomenthyl salicylate,
derivatives of benzophenone, for example 2-hydroxy-4-methoxybenzophenone, 2-hydroxy-4-methoxy-4′-methylbenzophenone, 2,2′-dihydroxy-4-methoxybenzophenone,
esters of benzylmalonic acid, for example di-2-ethylhexyl 4-methoxybenzmalonate, triazine derivatives, for example 2,4,6-trianilino-(p-carbo-2′-ethyl-1′-hexyloxy)-1,3,5-triazine, octyl triazone and those described in EP 1180359 and DE 2004/027475,
propane-1,3-diones, for example 1-(4-tert-butylphenyl)-3-(4′-methoxyphenyl) propane-1,3-dione.

Suitable water-soluble UVB light protection filters include:
2-phenylbenzimidazole-5-sulfonic acid and its alkali metal, alkaline earth metal, ammonium, alkylammonium, alkanolammonium and glucammonium salts,
sulfonic acid derivatives of benzophenone, for example 2-hydroxy-4-methoxybenzophenone-5-sulfonic acid and salts thereof,
sulfonic acid derivatives of 3-benzylidenecamphor, for example 4-(2-oxo-3-bornylidenemethyl)benzenesulfonic acid and 2-methyl-5-(2-oxo-3-bornylidene) sulfonic acid and salts thereof.

Suitable typical UVA light protection filters include in particular derivatives of benzoylmethane, for example 1-(4′-tert-butylphenyl)-3-(4′-methoxyphenyl) propane-1,3-dione or 1-phenyl-3-(4′-isopropylphenyl) propane-1,3-dione. It is of course also possible to use mixtures of UV-A and UV-B filters.

In addition to the recited soluble substances, insoluble pigments are also suitable for this purpose, namely finely dispersed metal oxides or salts, for example titanium dioxide, zinc oxide, iron oxide, aluminium oxide, cerium oxide, zirconium oxide, silicates (talc), barium sulfate and zinc stearate.

The particles here should have an average diameter of less than 100 nm, e.g. between 5 and 50 nm and in particular between 15 and 30 nm. They may be spherical in shape, although it is also possible to use particles that are ellipsoidal in shape or have a shape that deviates in some other way from spherical. A relatively new class of light protection filters is that of micronized organic pigments, for example 2,2′-methylenebis {6-(2H-benzotriazol-2-yl)-4-(1,1,3,3-tetramethylbutyl)phenol} having a particle size of <200 nm, which is obtainable for example as a 50% aqueous dispersion.

Further suitable UV light protection filters can be found in the review by P. Finkel in SOFW-Journal 122, 543 (1996).

In association with the formulations of the invention, said formulations preferably comprise lipophilic, hydrophobic UV light protection filter substances, in particular triazine derivatives.

Particular preference is given here to using as UV-B filters the UV light protection filter substances-2-ethylhexyl 2-cyano-3-phenylcinnamate, 2,4-bis {[4-(2-ethylhexyloxy)-2-hydroxy]phenyl}-6-(4-methoxyphenyl)-1,3,5-triazine, dioctylbutylamidotriazone, 2-hydroxy-4-methoxybenzophenone, 2-hydroxy-4-methoxy-4′-methylbenzophenone, 2,2′-dihydroxy-4-methoxybenzophenone, di-2-ethylhexyl 4-methoxybenzmalonate, 2,4,6-tris-[anilino-(p-carbo-2′-ethyl-1′-hexyloxy)]-1,3,5-triazine, 2,4-bis [5,1 (dimethylpropyl)benzoxazol-2-yl-(4-phenyl)imino]-6-(2-ethylhexyl)imino-1,3,5-triazine, 2,4-bis-{[4-(2-ethylhexyloxy)-2-hydroxy]phenyl}-6-(4-methoxyphenyl)-1,3,5-triazine and 2-[4,6-bis(2,4-dimethylphenyl)-1,3,5-triazin-2-yl]-5-(octyloxy) phenol.

UV-A filters used are preferably 1-(4′-tert-butylphenyl)-3-(4′-methoxyphenyl) propane-1,3-dione, 1-phenyl-3-(4′-isopropylphenyl) propane-1,3-dione.

Particularly preferred UV-A filters are 4-(tert-butyl)-4′-methoxydibenzoylmethane (CAS No. 70356 09-1), which is sold by DSM under the Parsol® 1789 brand and by Merck under the trade name Eusolex® 9020, and hydroxybenzophenones in accordance with DE 102004027475, particularly preferably hexyl 2-(4′-diethylamino-2′-hydroxybenzoyl)benzoate (also: aminobenzophenone), available under the name Uvinul A Plus from BASF.

Further preferred UV filter substances are further so-called broadband filters, i.e. filter substances that absorb both UV-A and UV-B radiation. Within this group, preference is given to using 2,2′-methylenebis(6-(2H-benzotriazol-2-yl)-4-(1,1,3,3-tetramethylbutyl) phenol), which is available under the trade name Tinosorb® M from Ciba Chemikalien GmbH, and 2-(2H-benzotriazol-2-yl)-4-methyl-6-[2-methyl-3-[1,3,3,3-tetramethyl-1-[(trimethylsilyl)oxy]disiloxanyl]propyl]phenol (CAS No.: 155633-54-8), which has the INCI name Drometrizole Trisiloxane.

In addition to the two groups of primary UV light protection filters mentioned above, it is also possible to employ secondary light stabilizers of the antioxidant type that arrest the photochemical reaction chain initiated when UV radiation penetrates the skin.

The cosmetic formulations of the invention comprise UV light protection filters preferably in an amount of 0.01% by weight to 15% by weight, preferably 0.05% by weight to 10% by weight, more preferably 0.1% by weight to 5% by weight, based on the overall formulation.

Preferably, the cosmetic formulations of the invention comprise a combination of two or more different UV light protection filters.

When both UVA and UVB light protection filters are used in a formulation of the invention, the weight ratio of these filters is preferably 1:2 to 1:4.

The formulations of the invention can further comprise at least one additional component selected from the following group:
emollients,
emulsifiers,
thickeners/viscosity regulators/stabilizers,
antioxidants,
hydrotropes (or polyols),
solids and fillers,
film formers,
pearlescence additives,
deodorant and antiperspirant active substances,
insect repellents,
preservatives,
conditioning agents,
perfumes,
dyes,
odour absorbers,
cosmetic active substances,
care additives,
superfatting agents,
solvents.

Substances that can be used as exemplary representatives of the individual groups are known to those skilled in the art and can be found for example in German patent application DE 102008001788.4. This patent application is hereby incorporated as reference and thus forms part of the disclosure.

As regards further optional components and the employed amounts of these components, express reference is made to the relevant handbooks known to those skilled in the art, for example K. Schrader, “Grundlagen und Rezepturen der Kosmetika” [Fundamentals and formulations of cosmetics], 2nd edition, pages 329 to 341, Hüthig Buch Verlag Heidelberg.

The amounts of each additive are guided by the intended use.

Typical frame formulations for particular applications are known prior art and are contained for example in the brochures of the manufacturers of the particular base materials and active ingredients. These existing formulations can generally be adopted unchanged. However, when necessary for adjustment and optimization, the desired modifications can be executed in a straightforward manner through simple tests.

The present invention further provides a sphingolipid of the general formula Ib

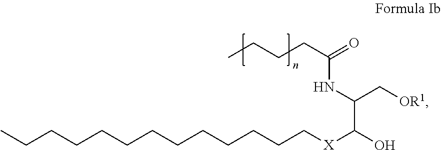

where
R¹ is H, phosphocholine, serine, ethanolamine or a sugar, preferably a sugar or H, more preferably H,
X is CH₂—HCOH, and
n 6,
or where
R¹ is H, phosphocholine, serine, ethanolamine or a sugar, preferably a sugar or H, more preferably H,
X is CH═CH or CH₂—HCOH, preferably CH₂—HCOH, and
n 5.

This sphingolipid of the general formula Ib has been found to boost the self tanning effect the most.

The present invention further provides a composition, preferably a cosmetic formulation comprising the sphingolipid of the general formula Ib. These cosmetic formulation according to the instant invention comprises the sphingolipid of the general formula Ib in the preferred ways as described above for component A) of the compositions according to the instant invention.

The present invention further provides a sphingolipid of the general formula Ib

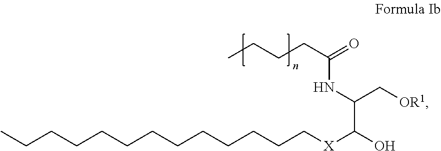

where
R¹ is H, phosphocholine, serine, ethanolamine or a sugar, preferably a sugar or H, more preferably H,
X is CH₂—HCOH, and
n 6,
or where
R¹ is H, phosphocholine, serine, ethanolamine or a sugar, preferably a sugar or H, more preferably H,
X is CH═CH or CH₂—HCOH, preferably CH₂—HCOH, and
n 5,
for use as a medicament.

The present invention further provides a sphingolipid of the general formula Ib

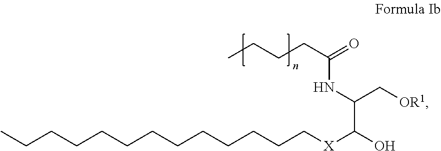

where
R¹ is H, phosphocholine, serine, ethanolamine or a sugar, preferably a sugar or H, more preferably H,
X is CH₂—HCOH, and
n 6,
or where
R¹ is H, phosphocholine, serine, ethanolamine or a sugar, preferably a sugar or H, more preferably H,
x is CH═CH or CH₂—HCOH, preferably CH₂—HCOH, and
n 5,
for the use in the treatment of acne, atopic dermatitis, psoriasis, herpes, warts, rosacea, skin cancer, pemphigus, alopecia, epidermolysis bullosa, scleroderma, ichthyosis, vitiligo and/or Raynaud's phenomenon.

The present invention further provides the use of at least one sphingolipid of the general formula I

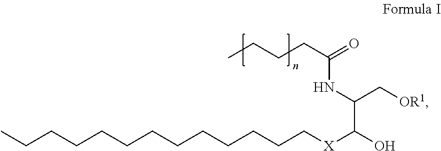

where
R¹ is H, phosphocholine, serine, ethanolamine or a sugar, preferably a sugar or H, more preferably H,
X is CH═CH, CH₂—CH₂ or CH₂—HCOH, preferably CH₂—HCOH, and
n is selected from an integer from 3 to 8, preferably from 6 and 7, most preferably 6, and/or at least one sphingoid base selected from the group of N-acetylphytosphingosine, phytosphingosine, sphingosine and sphinganine for enhancing the self tanning effect of a self tanning agent.

The present invention further provides the use of at least one composition according to the instant invention for tanning of a human skin.

Preferably the same preferred components A) and B) are used in the same preferred embodiments in the uses of the instant invention as described above for the composition according to the instant invention.

The uses of the instant invention are cosmetic, non-pharmaceutical uses.

The examples adduced hereinafter describe the present invention by way of example, without any intention that the invention, the scope of application of which is apparent from the entirety of the description and the claims, be restricted to the embodiments specified in the examples.

### EXAMPLES

### Example 1: Boosting of Self Tanning DHA

Experiments according to the invention are marked with *.

8 panels of 10 persons each are tested as follows.

A formulation containing DHA is applied to the left forearm, whereas a formulation containing DHA and a potential booster compound is applied to the right forearm.

| Formulation | 1 | 2 | 3 | 4 | 5* | 6* | 7* | 8* | 9* |
|---|---|---|---|---|---|---|---|---|---|
| TEGO ® Care 450 | 4.5 | 3 | 3 | 3 | 3 | 3 | 3 | 3 | 3 |
| Polyglyceryl-3 Methylglucose | | | | | | | | | |
| Distearate | | | | | | | | | |
| TEGOSOFT ® CT | 4.5 | 4.5 | 4.5 | 4.5 | 4.5 | 4.5 | 4.5 | 4.5 | 4.5 |
| Caprylic/Capric Triglyceride | | | | | | | | | |
| TEGOSOFT ® G 20 | 3 | 4.5 | 4.5 | 4.5 | 4.5 | 4.5 | 4.5 | 4.5 | 4.5 |
| Octyldodecanol | | | | | | | | | |
| Dihydroxyacetone | 4 | 4 | 4 | 4 | 4 | 4 | 4 | 4 | 4 |
| Ceramide 3 | | 0.1 | | | | | | | |
| Ceramide NS 22 | | | 0.1 | | | | | | |
| Ceramide NP C6 | | | | 0.1 | | | | | |
| Ceramide NS C13 | | | | | | | | | 0.1 |
| Ceramide NP C15 | | | | | 0.1 | | | | |
| Ceramide NP C17 | | | | | | 0.1 | | | |
| Phytosphingosine | | | | | | | 0.1 | | |
| Sphinganine | | | | | | | | 0.1 | |
| Water | | ad 100 | ad 100 | ad 100 | ad 100 | ad 100 | ad 100 | ad 100 | ad 100 |
| Verstatil ® PC | 0.7 | 0.7 | 0.7 | 0.7 | 0.7 | 0.7 | 0.7 | 0.7 | 0.7 |
| Phenoxyethanol, Caprylyl | | | | | | | | | |
| Glycol | | | | | | | | | |
| NaOH (10% aq)/lactic acid | 6.0 | 6.0 | 6.0 | 6.0 | 6.0 | 6.0 | 6.0 | 6.0 | 6.0 |
| (10% aq) ad pH | | | | | | | | | |

Ceramide NP C13: formula I with R¹═H, X═CH═CH, n = 5
Ceramide NP C15: formula I with R¹═H, X═CH₂—HCOH, n = 6
Ceramide NP C17: formula I with R¹═H, X═CH₂—HCOH, n = 7

Treatment is repeated daily over a period of 1 week (2.5 ml each side).

The tanning is assessed on a scale from 0 (no effect) to 5 (very strong tanning).

The average values are given in the table below.

| Panel | 1 | 2 | 3 | 4 | 5 | 6 | 7 | 8 |
|---|---|---|---|---|---|---|---|---|
| Formulation 1 | 1.9 | 2.1 | 2.1 | 2.0 | 2.1 | 2.1 | 1.9 | 1.9 |
| Formulation 2 | 1.9 | | | | | | | |
| Formulation 3 | | 2.1 | | | | | | |
| Formulation 4 | | | 2.0 | | | | | |
| Formulation 5* | | | | 2.9 | | | | |
| Formulation 6* | | | | | 2.6 | | | |
| Formulation 7* | | | | | | 2.3 | | |
| Formulation 8* | | | | | | | 2.4 | |
| Formulation 9* | | | | | | | | 2.8 |

As can be seen in the above only the ceramides with an odd number of carbon atoms and the spingoid bases are able to boost the self tanning effect.

### Example 2: Boosting of Self Tanning Erythrulose

Data is generated in analogy to example 1, but with erythrulose instead of DHA:

| Formulation | 9 | 10 | 11* | 12* |
|---|---|---|---|---|
| TEGOR Care 450 | 4.5 | 3 | 3 | 3 |
| Polyglyceryl-3 Methylglucose | | | | |
| Distearate | | | | |
| TEGOSOFT ® CT | 4.5 | 4.5 | 4.5 | 4.5 |
| Caprylic/Capric Triglyceride | | | | |
| TEGOSOFT ® G 20 | 3 | 4.5 | 4.5 | 4.5 |
| Octyldodecanol | | | | |
| Erythrulose | 4 | 4 | 4 | 4 |
| Ceramide 3 | | 0.1 | | |
| Ceramide NP C15 | | | 0.1 | |
| Ceramide NP C17 | | | | 0.1 |
| Water | | ad 100 | ad 100 | ad 100 |
| Verstatil ® PC | 0.7 | 0.7 | 0.7 | 0.7 |
| Phenoxyethanol, Caprylyl Glycol | | | | |
| NaOH (10% aq)/lactic acid | 6.0 | 6.0 | 6.0 | 6.0 |
| (10% aq) ad pH | | | | |

The average values are given in the table below.

| Panel | 8 | 9 | 10 |
|---|---|---|---|
| Formulation 9 | 1.2 | 1.3 | 1.2 |
| Formulation 10 | 1.3 | | |
| Formulation 11* | | 2.1 | |
| Formulation 12* | | | 1.9 |

As can be seen in the above only the ceramides with an odd number of carbon atoms are able to boost the self tanning effect.

### Example 3: Example Formulations

### Face Cream I

Phase

| | | | | | | | | | |
|---|---|---|---|---|---|---|---|---|---|
| A | TEGO ® Care 450 (Polyglyceryl-3 | 3.0 | 3.0 | 3.0 | 3.0 | 3.0 | 3.0 | 3.0 | 3.0 |
| | Methylglucose Distearate) | | | | | | | | |
| | TEGIN ® M Pellets MB (Glyceryl | 2.0 | 2.0 | 2.0 | 2.0 | 2.0 | 2.0 | 2.0 | 2.0 |
| | Stearate) | | | | | | | | |
| | TEGO ® Alkanol 1618 MB | 1.0 | 1.0 | 1.0 | 1.0 | 1.0 | 1.0 | 1.0 | 1.0 |
| | (Cetearyl Alcohol) | | | | | | | | |
| | TEGOSOFT ® OS | 8.0 | 8.0 | 8.0 | 8.0 | 8.0 | 8.0 | 8.0 | 8.0 |
| | (Ethylhexylstearate) | | | | | | | | |
| | TEGOSOFT ® OER MB (Oleyl | 5.0 | 5.0 | 5.0 | 5.0 | 5.0 | 5.0 | 5.0 | 5.0 |
| | Erucate) | | | | | | | | |
| | TEGOSOFT ® DC MB (Decyl | 6.0 | 6.0 | 6.0 | 6.0 | 6.0 | 6.0 | 6.0 | 6.0 |
| | Cocoate) | | | | | | | | |
| | Ceramide NP C15 | 0.1 | | 0.1 | | 0.1 | 0.1 | 0.1 | 0.1 |
| | Ceramide NP C17 | | 0.1 | | 0.1 | | | | |
| | SPHINOX ® Defenda | | | 0.1 | 0.1 | | | | |
| | (Hydroxybutyroyl | | | | | | | | |
| | Phytosphingosine) | | | | | | | | |
| B | Glycerin | 3.0 | 3.0 | 3.0 | 3.0 | 3.0 | 3.0 | 3.0 | 3.0 |
| | Water | 70.9 | 69.9 | 70.8 | 69.8 | 70.8 | 70.8 | 68.9 | 68.9 |
| | HyaCare ® (Sodium Hyaluronate) | | | | | 0.1 | | | |
| | HyaCare ® 50 (Hydrolyzed | | | | | | 0.1 | | |
| | Hyaluronic Acid) | | | | | | | | |
| | Dihydroxyacetone | | 1.0 | | 1.0 | | | | |
| C | TEGO ® Pep 4-17 (Tetrapeptide- | | | | | | | 2.0 | |
| | 21, Glycerin, Butylene Glycol, | | | | | | | | |
| | Water) | | | | | | | | |
| | TEGO ® Pep UP (Tetrapeptide- | | | | | | | | 2.0 |
| | 4, Glycerin, Water) | | | | | | | | |
| D | Verstatil ® PC (Phenoxyethanol, | 1.0 | 1.0 | 1.0 | 1.0 | 1.0 | 1.0 | 1.0 | 1.0 |
| | Caprylyl Glycol) | | | | | | | | |

Preparation: Heat phase A and B to 85° C. Add phase A to B with stirring. Homogenise. Cool down while stirring to 40° C. Add phase C and D. Cool down while stirring to 30° C.

### Face Cream II

Phase

| | | | | | | | | | |
|---|---|---|---|---|---|---|---|---|---|
| A | TEGO ® Care 450 (Polyglyceryl-3 | 3.0 | 3.0 | 3.0 | 3.0 | 3.0 | 3.0 | 3.0 | 3.0 |
| | Methylglucose Distearate) | | | | | | | | |
| | TEGIN ® M Pellets MB (Glyceryl | 2.0 | 2.0 | 2.0 | 2.0 | 2.0 | 2.0 | 2.0 | 2.0 |
| | Stearate) | | | | | | | | |
| | TEGO ® Alkanol 1618 MB | 1.0 | 1.0 | 1.0 | 1.0 | 1.0 | 1.0 | 1.0 | 1.0 |
| | (Cetearyl Alcohol) | | | | | | | | |
| | TEGOSOFT ® OS (Ethylhexylstearate) | 8.0 | 8.0 | 8.0 | 8.0 | 8.0 | 8.0 | 8.0 | 8.0 |
| | TEGOSOFT ® OER MB (Oleyl | 5.0 | 5.0 | 5.0 | 5.0 | 5.0 | 5.0 | 5.0 | 5.0 |
| | Erucate) | | | | | | | | |
| | TEGOSOFT ® DC MB (Decyl | 6.0 | 6.0 | 6.0 | 6.0 | 6.0 | 6.0 | 6.0 | 6.0 |
| | Cocoate) | | | | | | | | |
| | Ceramide NP C15 | 0.1 | | 0.1 | | 0.1 | 0.1 | 0.1 | 0.1 |
| | Ceramide NP C13 | | 0.1 | | 0.1 | | | | |
| | SPHINOX ® Defenda | | | 0.1 | 0.1 | | | | |
| | (Hydroxybutyroyl | | | | | | | | |
| | Phytosphingosine) | | | | | | | | |
| B | Glycerin | 3.0 | 3.0 | 3.0 | 3.0 | 3.0 | 3.0 | 3.0 | 3.0 |
| | Water | 70.9 | 70.9 | 70.8 | 70.8 | 70.8 | 70.8 | 68.9 | 68.9 |
| | HyaCare ® (Sodium Hyaluronate) | | | | | 0.1 | | | |
| | HyaCare ® 50 (Hydrolyzed | | | | | | 0.1 | | |
| | Hyaluronic Acid) | | | | | | | | |
| C | RHEASOME ® Ceraboost | | | | | | | 4.0 | |
| | (Water/Aqua; Glycerin; Lecithin; | | | | | | | | |
| | Glycolipids; Ceramide NP; | | | | | | | | |
| | Phytosphingosine; Cholesterol; | | | | | | | | |
| | Benzyl Alcohol; Benzoic Acid; | | | | | | | | |
| | Dehydroacetic Acid; Sodium | | | | | | | | |
| | Benzoate; Tocopherol) | | | | | | | | |
| | InnuMax ® Advanced Retinol | | | | | | | | 5.0 |
| | (Water/Aqua; Caprylic/Capric | | | | | | | | |
| | Triglyceride; Glycerin; | | | | | | | | |
| | Polysorbate 20; Retinol; Inulin | | | | | | | | |
| | Lauryl Carbamate; | | | | | | | | |
| | Behentrimonium Chloride; | | | | | | | | |
| | Sucrose Laurate) | | | | | | | | |
| D | Verstatil ® PC (Phenoxyethanol, | 1.0 | 1.0 | 1.0 | 1.0 | 1.0 | 1.0 | 1.0 | 1.0 |
| | Caprylyl Glycol) | | | | | | | | |

Preparation: Heat phase A and B to 85° C. Add phase A to B with stirring. Homogenize. Cool down while stirring to 40° C. Add phase C and D. Cool down while stirring to 30° C.

### Face Cream III

Phase

| | | | | | | | | | |
|---|---|---|---|---|---|---|---|---|---|
| A | TEGO ® Care 450 (Polyglyceryl-3 | 3.0 | 3.0 | 3.0 | 3.0 | 3.0 | 3.0 | 3.0 | 3.0 |
| | Methylglucose Distearate) | | | | | | | | |
| | TEGIN ® M Pellets MB (Glyceryl | 2.0 | 2.0 | 2.0 | 2.0 | 2.0 | 2.0 | 2.0 | 2.0 |
| | Stearate) | | | | | | | | |
| | TEGO ® Alkanol 1618 MB | 1.0 | 1.0 | 1.0 | 1.0 | 1.0 | 1.0 | 1.0 | 1.0 |
| | (Cetearyl Alcohol) | | | | | | | | |
| | TEGOSOFT ® OS | 8.0 | 8.0 | 8.0 | 8.0 | 8.0 | 8.0 | 8.0 | 8.0 |
| | (Ethylhexylstearate) | | | | | | | | |
| | TEGOSOFT ® OER MB (Oleyl | 5.0 | 5.0 | 5.0 | 5.0 | 5.0 | 5.0 | 5.0 | 5.0 |
| | Erucate) | | | | | | | | |
| | TEGOSOFT ® DC MB (Decyl | 6.0 | 6.0 | 6.0 | 6.0 | 6.0 | 6.0 | 6.0 | 6.0 |
| | Cocoate) | | | | | | | | |
| | Ceramide NP C15 | 0.1 | | 0.1 | | 0.1 | 0.1 | 0.1 | 0.1 |
| | Ceramide NS C13 | | 0.1 | | 0.1 | | | | |
| | SPHINOX ® Defenda | | | 0.1 | 0.1 | | | | |
| | (Hydroxybutyroyl | | | | | | | | |
| | Phytosphingosine) | | | | | | | | |
| B | Glycerin | 3.0 | 3.0 | 3.0 | 3.0 | 3.0 | 3.0 | 3.0 | 3.0 |
| | Water | 70.9 | 70.9 | 70.8 | 70.8 | 70.8 | 70.8 | 68.9 | 68.9 |
| | HyaCare ® (Sodium Hyaluronate) | | | | | 0.1 | | | |
| | HyaCare ® 50 (Hydrolyzed | | | | | | 0.1 | | |
| | Hyaluronic Acid) | | | | | | | | |
| C | SK-INFLUX ® V MB (Ceramide | | | | | | | 5.0 | |
| | NP; Ceramide AP; Ceramide | | | | | | | | |
| | EOP; Phytosphingosine; | | | | | | | | |
| | Cholesterol; Sodium Lauroyl | | | | | | | | |
| | Lactylate; Carbomer; Xanthan | | | | | | | | |
| | Gum) | | | | | | | | |
| | SK-INFLUX ® Evolve MB | | | | | | | | 5.0 |
| | (Ceramide NP; Ceramide AP; | | | | | | | | |
| | Ceramide EOP; | | | | | | | | |
| | Phytosphingosine; Cholesterol; | | | | | | | | |
| | Behenic Acid; Polyglyceryl-10 | | | | | | | | |
| | Stearate; Polyglyceryl-6 | | | | | | | | |
| | Behenate; Cetearyl Alcohol; | | | | | | | | |
| | Glyceryl Stearate; Glycerin; | | | | | | | | |
| | Sodium Cetearyl Sulfate; Triethyl | | | | | | | | |
| | Citrate; Sodium Levulinate; | | | | | | | | |
| | Potassium Sorbate; Water/Aqua) | | | | | | | | |
| D | Verstatil ® PC (Phenoxyethanol, | 1.0 | 1.0 | 1.0 | 1.0 | 1.0 | 1.0 | 1.0 | 1.0 |
| | Caprylyl Glycol) | | | | | | | | |

Preparation: Heat phase A and B to 85° C. Add phase A to B with stirring. Homogenise. Cool down while stirring to 40° C. Add phase C and D. Cool down while stirring to 30° C.

### O/W Lotion, Skin Fluid

| Phase | Ingredients (INCI) | | | | | | |
|---|---|---|---|---|---|---|---|
| A | TEGO ® Care PBS 6 MB (Polyglyceryl-6 | 3.0 | 3.0 | 3.0 | 3.0 | 3.0 | 3.0 |
| | Stearate (and) Polyglyceryl-6 Behenate) | | | | | | |
| | TEGO ® Alkanol 1618 MB (Cetearyl Alcohol) | 1.0 | 1.0 | 1.0 | 1.0 | 1.0 | 1.0 |
| | TEGIN ® M Pellets MB (Glyceryl Stearate) | 1.0 | 1.0 | 1.0 | 1.0 | 1.0 | 1.0 |
| | TEGOSOFT ® MM (Myristyl Myristate) | 1.0 | 1.0 | 1.0 | 1.0 | 1.0 | 1.0 |
| | TEGOSOFT ® OS (Ethylhexyl Stearate) | 5.0 | 5.0 | 5.0 | 5.0 | 5.0 | 5.0 |
| | Isopropyl Myristate | 3.5 | 3.5 | 3.5 | 3.5 | 3.5 | 3.5 |
| | ABIL ® 350 (Dimethicone) | 2.0 | 2.0 | 2.0 | 2.0 | 2.0 | 2.0 |
| | Mineral Oil (Paraffinum Liquidum) | 5.0 | 5.0 | 5.0 | 5.0 | 5.0 | 5.0 |
| | Ceramide NP C15 | 0.1 | | 0.1 | 0.1 | | |
| | Ceramide NP C17 | | 0.1 | | | 0.1 | 0.1 |
| B | SK-INFLUX ® V MB (Ceramide NP; Ceramide | | | 5.0 | 5.0 | 5.0 | 5.0 |
| | AP; Ceramide EOP; Phytosphingosine; | | | | | | |
| | Cholesterol; Sodium Lauroyl Lactylate; | | | | | | |
| | Carbomer; Xanthan Gum | | | | | | |
| | Water | 63.9 | 62.9 | 54.9 | 56.9 | 54.4 | 56.4 |
| | Dihydroxyacetone | | 1.0 | | | | |
| | Erythrulose | | | | | 0.5 | 0.5 |
| C | Glycerin | 3.0 | 3.0 | 3.0 | 3.0 | 3.0 | 3.0 |
| | Keltrol CG SFT (Xanthan Gum) | 0.5 | 0.5 | 0.5 | 0.5 | 0.5 | 0.5 |
| | Water | 10.0 | 10.0 | 10.0 | 10.0 | 10.0 | 10.0 |
| D | TEGO ® Natural Betaine (Betaine) | | | 2.0 | | 2.0 | |
| | Water | | | 2.0 | | 2.0 | |
| | LACTIL ® (Sodium Lactate; Sodium PCA; | | | | 2.0 | | 2.0 |
| | Glycine; Fructose; Urea; Niacinamide; Inositol; | | | | | | |
| | Sodium Benzoate; Lactic Acid) | | | | | | |
| E | Verstatil ® TBO (Triethyl Citrate; Caprylyl | 1.0 | 1.0 | 1.0 | 1.0 | 1.0 | 1.0 |
| | Glycol; Benzoic Acid) | | | | | | |

### Preparation:

Heat phase A and B to 85° C. Add phase A to B with stirring. Homogenise. Cool with gentle stirring to approx. 40° C. and add phase C. Homogenise for a short time. Add phase D and E step by step.

### Enjoy the Sun Lotion, SPF 50

| Phase | Ingredients (INCI) | | |
|---|---|---|---|
| A | TEGO ® Care PBS 6 (Polyglyceryl-6 Stearate (and) Polyglyceryl-6 | 4.00 | 4.00 |
| | Behenate) | | |
| | dermofeel sensolv (Isoamyl Laurate) | 8.00 | 8.00 |
| | TEGIN ® M Pellets MB (Glyceryl Stearate | 0.25 | 0.25 |
| | TEGO ® Alkanol 1618 MB (Cetearyl Alcohol) | 0.25 | 0.25 |
| | dermofeel ® Toco 70 non GMO (Tocopherol; Helianths Anuus | 0.20 | 0.20 |
| | (Sunflower) Seed Oil) | | |
| | Bis-Ethyhexyloxyphenol Methoxyphenyl Triazine | 6.00 | 6.00 |
| | Diethylamino Hydroxybenzoyl Hexyl Benzoate | 6.00 | 6.00 |
| | Ethylhexyl Salicylate | 5.00 | 5.00 |
| | Ethylhexyl Triazone | 2.00 | 2.00 |
| | Ceramide NP C15 | 0.10 | |
| | Ceramide NP C13 | | 0.10 |
| B | Water | 48.45 | 48.45 |
| | Glycerin | 3.00 | 3.00 |
| | Disodium EDTA | 0.05 | 0.05 |
| C | Keltrol CG SFT (Xanthan Gum) | 0.50 | 0.50 |
| D | Water | 10.67 | 10.67 |
| | Euxolex 232 (Phnylbenzimidazole Sulfonic Acid) | 3.00 | 3.00 |
| | Tromethamine | 1.33 | 1.33 |
| E | Perfume | q.s | q.s |
| | Tromethamine, 30% in water | q.s. | q.s. |
| F | Verstatil ® BOB (Benzyl Alcohol; Caprylyl Glycol; Benzoic Acid) | 1.20 | 1.20 |

### Preparation:

Heat phase A and B separately to approx. 85° C. Add phase A to B. Homogenize. Cool with gentle stirring to approx . . . 50° C., then add phase C. Homogenise. Cool with gentle stirring to approx . . . 35° C., then add phase D, E and F. Homogenise. Adjust the pH to 6.5-7.0 with phase E.

### Microemulsion

| Phase | Ingredients (INCI) | | |
|---|---|---|---|
| A | Water | 91.9 | 91.9 |
| B | VARISOFT ® PATC (Palmitamidopropyltrimonium Chloride) | 2.5 | 2.5 |
| | Ceramide NP C15 | 0.1 | |
| | Ceramide NS C13 | | 0.1 |
| | Glycerin | 3.0 | 3.0 |
| C | LACTIL ® (Sodium Lactate; Sodium PCE; Fructose; Urea; Niacinamide; | 2.0 | 2.0 |
| | Inositol; Sodium Benzoate; Lactic Acid) | | |
| D | TEGO ® Cosmo C 100 (Creatine) | 0.5 | 0.5 |
| E | Verstatil ® SL (Aqua; Sodium Levulinate; Potassium Sorbate) | 2.0 | 2.0 |
| F | Lactic Acid (10% in water) | q.s | q.s |

### Preparation:

Heat phase B to appr. 85° C. until the Ceramide NP C15 resp. Ceramide NP C17 is solubilized clearly. Add phase B to phase A (20-22° C.) slowly while stirring. Add phase C, D and E. Adjust pH with phase F to PH 5-5.5.

### Facial ON

| Phase | Ingredients (INCI) | | | |
|---|---|---|---|---|
| A | dermofeel ® sensolv MB (Isoamyl Laurate) | 93.73 | 93.73 | 93.73 |
| | dermofeel ® Toco 70 non GMO (Tocopherol; Helianthus Annuus | 0.20 | 0.20 | 0.20 |
| | (Sunflower) Seed Oil) | | | |
| | Perfume | 1.00 | 1.00 | 1.00 |
| | TEGOSOFT ® DC (Decyl Cocoate) | 1.00 | 1.00 | 1.00 |
| | Argan Oil, cold pressed (Argania Spinosa Kernel Oil) | 1.00 | 1.00 | 1.00 |
| | Broccoli Seed Oil, cold pressed (Brassica Olearacea Italica | 1.00 | 1.00 | 1.00 |
| | Seed Oil) | | | |
| | Carrot Oil (Daucus Carota Sativa Juice; Sesamum Indicum | 0.50 | 0.50 | 0.50 |
| | Seed Oil) | | | |
| | Ceramide NP C15 | 0.05 | | |
| | Ceramide NP C13 | | 0.05 | |
| | Ceramide NS C13 | | | 0.5 |
| | Coconut Oil, refined, organic (Cocos Nucifera Oil) | 1.00 | 1.00 | 1.00 |
| | Seabuckthorn Pulp Oil (Hippophae Rhamnoides Fruit Oil) | 0.02 | 0.02 | 0.02 |
| | dermosoft ® GMCY (Glyceryl Caprylate) | 0.50 | 0.50 | 0.50 |

### Preparation

Mix ingredients in given order and heat up to 85° C. Stir slowly until a clear solution results. Cool down.

### Facial Cleanser

| Phase | Ingredients (INCI) | | |
|---|---|---|---|
| A | TEGOSOFT ® PC 31 (Polyglyceryl-3 Caprate) | 2.50 | 2.50 |
| | TEGO ® Solve 61 MB (Polyglyceryl-6 Caprate; Polyglyceryl-3 Cocoate; | 2.50 | 2.50 |
| | Polyglyceryl-4 Caprate; Polyglyceryl-6 Ricinoleate) | | |
| B | Ceramide NP C15 | 0.07 | |
| | Ceramide NP C13 | | 0.07 |
| | SPHINGONY ® (Sphinganine) | 0.10 | 0.10 |
| | dermosoft ® Pentiol eco (Pentylene Glycol) | 5.50 | 5.50 |
| | TAGAT CH 40 (PEG-40 Hydrogenated Castor Oil) | 6.00 | 6.00 |
| C | Water | 83.13 | 83.13 |
| | Keltrol CG-SFT (Xanthan Gum) | 0.20 | 0.20 |
| D | Lactic Acid (10% in water) | q.s. | q.s. |

### Preparation

Heat phase B up to 85° C. with stirring. Cool down to room temperature. Add phase A to B with gentle stirring. Prepare phase C by dispersing Xanthan Gum in water. Add phase C slowly with stirring, step by step. Adjust pH with phase D between 5.0-5.5.

### Shampoo

| Phase | Ingredients (INCI) | | |
|---|---|---|---|
| A | Sodium Laureth Sulfate, 28% | 32.00 | 32.00 |
| | dermofeel ® sensolv MB (Isoamyl Laurate) | 1.00 | 1.00 |
| | dermofeel ® G10 LW 70 MB (Polyglyceryl-10 Laurate; Aqua; Citric Acid) | 4.00 | 4.00 |
| | Ceramide NP C15 | 0.03 | |
| | Ceramide NP C13 | | 0.03 |
| | TEGO ® Betain P 50 SG (Cocoamidopropyl Betaine) | 10.00 | 10.00 |
| | dermofeel ® quadegra (Aqua; Starch Hydroxypropyl trimonium Chloride; | 1.50 | 1.50 |
| | Urea; Sodium Lactate; Lactic Acid; Sodium Chloride; Glycerin; Levulinic | | |
| | Acid; p-Anisic Acid) | | |
| | Water | | |
| | Verstatil ® BP (Phenoxyethanol; Benzoic Acid) | 0.80 | 0.80 |
| | ANTIL ® 200 MB (PEG-200 Hydrogenated Glyceryl Palmate; PEG-7 | 4.00 | 4.00 |
| | Glyceryl Cocoate) | | |

### Preparation:

Heat Sodium Laureth Sulfate, dermofeel sensolv MB, dermofeel G10 LW 70 MB and Ceramide NP C15 resp. Ceramide NP C17 to 85° C. until the solution is clear. Add the other ingredients step by step in the given order. Cool down while stirring to 30° C.

### Oral Care (Mouth Wash)

| Phase | Ingredients (INCI) | | |
|---|---|---|---|
| A | RHEANCE ® One (Glycolipids) | 0.20 | 0.20 |
| | Ceramide NP C15 | 0.02 | |
| | Ceramide NP C13 | | 0.02 |
| | Flavor Mintfresh | 0.20 | 0.20 |
| | Water | 93.51 | 93.51 |
| | Sodium Saccharine 450 | 0.07 | 0.07 |
| | Sorbitol, 70% | 4.00 | 4.00 |
| | Verstatil ® SL non GMO (Aqua; Sodium Levulinate; Potassium Sorbate) | 2.00 | 2.00 |

### Preparation

Heat RHEANCE One and Ceramide NP C15 resp. Ceramide NP C17 to 85° C. Cool down to 40° C. Dissolve the flavour. Add the water slowly while stirring, then the remaining ingredients in the given order.

### Deo Formulation (Natural Milky Deo Roll-On

| Phase | Ingredients (INCI) | | | |
|---|---|---|---|---|
| A | Water | 80.53 | 80.53 | 80.53 |
| | Glycerin | 5.00 | 5.00 | 5.00 |
| | Trisodium Citrate Dihydrate (Sodium Citrate) | 0.30 | 0.30 | 0.30 |
| | Xanthan Gum FNCSP-PC (Xanthan Gum) | 0.90 | 0.90 | 0.90 |
| | Genuvisco Carrageenan CG131 (Carrageenan) | 0.10 | 0.10 | 0.10 |
| B | dermosoft ® decalact delo MB (Sodium Caproyl/Lauroyl | 0.30 | 0.30 | 0.30 |
| | Lactylate; Triethyl Citrate; Salvia Officinalis (Sage) Oil) | | | |
| C | dermofeel ® GSC SG (Glyceryl Stearate Citrate) | 0.40 | 0.40 | 0.40 |
| | Apricot Kernel Oil, refined (Prunus Armeniaca Kernel Oil) | 0.50 | 0.50 | 0.50 |
| | Sunflower Oil, pressed, organic (Helianthus annuus seed oil) | 0.50 | 0.50 | 0.50 |
| | TEGODEO ® PY 88 G (Zinc Ricinoleate) | 1.00 | 1.00 | 1.00 |
| | dermofeel ® TEC eco (Triethyl Citrate) | 5.00 | 5.00 | 5.00 |
| | dermosoft ® G 3 CY MB (Polyglyceryl-3 Caprylate) | 1.00 | 1.00 | 1.00 |
| | Ceramide NP C15 | 0.10 | | |
| | Ceramide NP C13 | | 0.10 | |
| | Ceramide NS C13 | | | 0.10 |
| D | Perfume | 0.30 | 0.30 | 0.30 |
| | dermosoft ® 7GA MB (Aqua; Glycerin; Heptylglycerin; Sodium | 2.60 | 2.60 | 2.60 |
| | Anisate) | | | |
| E | Citric Acid (20% in water) | 1.47 | 1.47 | 1.47 |

### Preparation

Heat phase A up to 80° C. and disperse phase B in A. Heat phase C up to 85° C. separately. Add phase C into phase A/B. Cool down to 35° C. and add phase D in the given order under slowly stirring. Add phase E to adjust the pH value.

### Shower Gel

| Phase | Ingredients (INCI) | | |
|---|---|---|---|
| A | Sodium Laureth Sulfate, 28% | 37.00 | 37.00 |
| | Verstatil ® PC (Phenoxyethanol; Caprylyl Glycol) | 1.00 | 1.00 |
| | ANTIL ® Soft SC MB (Sorbitan Sesquicaprylate) | 1.00 | 1.00 |
| | Ceramide NP C15 | 0.05 | |
| | Ceramide NP C13 | | 0.05 |
| | Water | 42.55 | 42.55 |
| | REWOTERIC ® AM C MB (Sodium Cocoamphoacetate) | 9.00 | 9.00 |
| | TEGO ® Betain 810 MB (Capryl/Capramidopropyl Betaine) | 7.60 | 7.60 |
| | Citric Acid (30% in water) | 1.30 | 1.30 |
| | REWODERM ® LI S 80 MB (PEG-200 Hydrogenated Glyceryl Palmate; | 0.50 | 0.50 |
| | PEG-7 Glyceryl Cocoate) | | |
| | Sodium Chloride | 0.20 | 0.20 |

### Preparation

Heat Sodium Laureth Sulfate, Verstatil PC, ANTIL SOFT SC MB and Ceramide NP C15 resp. Ceramide NP C17 to 85° C. Add the other ingredients slowly step by step while stirring.

### Facial Masque

| Phase | Ingredients (INCI) | | | |
|---|---|---|---|---|
| A | TEGO ® Care PBS 6 (Polyglyceryl-6 Stearate (and) | 4.00 | 4.00 | 4.00 |
| | Polyglyceryl-6 Behenate) | | | |
| | Stearyl Alcohol | 2.00 | 2.00 | 2.00 |
| | TEGIN ® M Pellets MB (Glyceryl Stearate) | 1.00 | 1.00 | 1.00 |
| | TEGOSOFT CR MB (Cetyl Ricinoleate) | 2.00 | 2.00 | 2.00 |
| | TEGOSOFT ® ® APM (PPG-3 Myristyl Ether) | 12.0 | 12.0 | 12.0 |
| | TEGOSOFT ® OP (Ethylhexyl Palmiate) | 10.00 | 10.00 | 10.00 |
| | Ceramide NP C15 | 0.20 | | |
| | Ceramide NP C17 | | 0.20 | 0.20 |
| | dermosoft ® OMP (Methylpropanediol; Caprylyl Glycol; | 3.50 | 3.50 | 3.50 |
| | Phenylpropanol) | | | |
| B | Water | 37.50 | 36.50 | 37.00 |
| | Dihydroxyacetone | | 1.00 | |
| | Erythrulose | | | 0.50 |
| C | Sclerotium Gum | 0.53 | 0.53 | 0.53 |
| | Glycerin | 5.00 | 5.00 | 5.00 |
| | Keltrol CG-SFT (Xanthan Gum) | 0.27 | 0.27 | 0.27 |
| D | TEGO ® Renew HA Malto (Maltobionic Acid) | 1.00 | 1.00 | 1.00 |
| | TEGO ® Renew HA Lacto (Lactobionic Acid) | 1.00 | 1.00 | 1.00 |
| | Water | 20.00 | 20.00 | 20.00 |
| E | Citric Acid (10% in water) | q.s. | q.s. | q.s. |

### Preparation

Heat phase A and B to 85° C. Add phase A to B with stirring. Homogenise. Cool with gentle stirring to 50° C. Add phase C at about 50° C. Homogenize again for a short time. Cool with gentle stirring to appr. 35° C. Prepare phase D. Add phase D with stirring. Adjust pH with phase E to 4.0-5.0.

### Conditioner for Hair

| Phase | Ingredients (INCI) | | |
|---|---|---|---|
| A | TEGO ® Alkanol 1618 MB (Cetearyl Alcohol) | 6.00 | 6.00 |
| | VARISOFT ® BT 85 Pellets (Behentrimonium Chloride) | 2.50 | 2.50 |
| | VARISOFT ® EQ 100 (Bis-(Isosearoyl/Oleoyl Isopropyl) Dimonium | 1.00 | 1.00 |
| | Methosulfate) | | |
| | Hairflux ® (Olea Europaea (Olive) Fruit Oil; | 1.00 | 1.00 |
| | Ricinus Communis (Castor) Seed Oil; Ceramide NG | | |
| | Ceramide NP C15 | 0.10 | |
| | Ceramide NP C13 | | 0.10 |
| B | ABIL ® Quat 3272 (Quaternium-80) | 2.00 | 2.00 |
| | Glycerin | 2.00 | 2.00 |
| | Water | 84.40 | 84.40 |
| C | Verstatil ® PC (Phenoxyethanol; Caprylyl Glycol) | 1.00 | 1.00 |

### Preparation

Heat phase A and B to 85° C. Add phase B to phase A without stirring. Homogenise. Cool down to 30° C. while stirring.

### W/O Emulsion

| Phase | Ingredients (INCI) | | |
|---|---|---|---|
| A | ABIL ® EM 90 (Cetyl PEG/PPG-10/1 Dimethicone) | 2.00 | 2.00 |
| | Paracera W 80 (Microcristalline Wax) | 0.50 | 0.50 |
| | Hydrogenated Castor Oil | 0.50 | 0.50 |
| | Octyldodecanol | 7.00 | 7.00 |
| | TEGOSOFT ® CT (Caprylic/Capric Triglyceride) | 7.00 | 7.00 |
| | TEGOSOFT ® DC MB (Decyl Cocoate) | 7.00 | 7.00 |
| | Ceramide NP C15 | 0.10 | |
| | Ceramide NP C13 | | 0.10 |
| B | Glycerin | 3.00 | 3.00 |
| | Sodium Chloride | 0.50 | 0.50 |
| | Water | 71.40 | 71.40 |
| C | Euxyl PE 9010 (Phenoxyethanol; Ethylhexylglycerin) | 1.00 | 1.00 |

### Preparation

Heat phase A to approx. 85° C. Add phase B (80° C. or room temperature) slowly while stirring. Homogenise for a short time. Cool with gentle stirring below 30° C. and homogenise again.

### 20.1 Cream for Treatment of Sensitive Skin

| | |
|---|---|
| A | 1.50 wt.-% TEGO ® Care 450 MB (Polyglyceryl-3 Methylglucose Distearate) |
| | 0.50 wt.-% ABIL ® Care XL 80 MB (Bis-PEG/PPG-20/5 PEG/PPG-20/5 Dimethicone; |
| | Methoxy PEG/PPG-25/4 Dimethicone; Caprylic/Capric Triglyceride) |
| | 1.50 wt.-% Stearyl Alcohol |
| | 2.50 wt.-% TEGIN ® M Pellets MB (Glyceryl Stearate) |
| | 6.50 wt.-% TEGOSOFT ® M (Isopropyl Myristate) |
| | 4.80 wt.-% TEGOSOFT ® APM (PPG-3 Myristyl Ether) |
| | 0.50 wt.-% TEGOSOFT ® MM MB (Myristyl Myristate) |
| | 0.10 wt.-% Ceramide NP 15 |
| | 0.10 wt.-% SPHINGOKINE NP (Caprooyl Phytosphingosine) |
| B | 1.00 wt.-% TEGO ® Natural Betaine (Betaine) |
| | 3.00 wt.-% Glycerin |
| | ad 100 wt.-% Water |
| C | 0.20 wt.-% Carbomer |
| | 0.10 wt.-% Xanthan Gum |
| | 0.80 wt.-% TEGOSOFT ® M (Isopropyl Myristate) |
| D | q.s. Sodium Hydroxide (10% in water) |
| | 1.00 wt.-% Verstatil ® PC (Phenoxyethanol; Caprylyl Glycol) |
| | q.s. Perfume |

### 20.2 Facial Mask for Reactive Skin

| | |
|---|---|
| A | 0.50 wt.-% ABIL ® Care XL 80 MB (Bis-PEG/PPG-20/5 PEG/PPG-20/5 Dimethicone; |
| | Methoxy PEG/PPG-25/4 Dimethicone; Caprylic/Capric Triglyceride) |
| | 1.50 wt.-% ABIL ® 350 (Dimethicone) |
| | 4.00 wt.-% TEGOSOFT ® OS (Ethylhexyl Stearate) |
| | 4.00 wt.-% TEGOSOFT ® CT MB (Caprylic/Capric Triglyceride) |
| | 0.10 wt.-% Ceramide NP 15 |
| B | 0.60 wt.-% TEGO ® Carbomer 341 ER (Acrylates/C10-30 Alkyl Acrylate Crosspolymer) |
| | 0.10 wt.-% HyaCare ® (Sodium Hyaluronate) |
| | 0.10 wt.-% HyaCare ® 50 (Hydrolyzed Hyaluronic Acid) |
| | 5.00 wt.-% Glycerin |
| | ad 100 wt.-% Water |
| C | 1.00 wt.-% Verstatil ® PC (Phenoxyethanol; Caprylyl Glycol) |
| D | q.s. Sodium Hydroxide (10% in water) |

### 20.3 Microbiome Balancing Serum

| | |
|---|---|
| A | 0.30 wt.-% TEGO ® Carbomer 341 ER (Acrylates/C10-30 Alkyl Acrylate Crosspolymer) |
| B | ad 100 wt.-% Water |
| C | q.s. Sodium Hydroxide (10% in water) |
| D | 1.00 wt.-% TEGOR SML 20 MB (Polysorbate 20) |
| E | 0.10 wt.-% Ceramide NP 15 |
| | 3.00 wt.-% InuMax Advanced Retinol (Aqua; Caprylic/Capric Triglyceride; Glycerin; |
| | Polysorbate 20; Retinol; Inulin Lauryl Carbamate; Behentrimonium Chloride; Sucrose |
| | Laurate; BHT; BHA; Phenoxyethanol; Benzoic Acid; Dehydroacetic Acid; |
| | Ethylhexylglycerin; Disodium EDTA) |
| | 2.00 wt.-% Verstatil ® PC (Phenoxyethanol; Caprylyl Glycol) |

### 20.4 Skin Tone Balancing Cream

| | |
|---|---|
| A | ad 100 wt.-% Water, deionized |
| | 2.00 wt.-% Glycerin 99.5%, Ph. Eur. (Glycerin) |
| | 0.10 wt.-% dermofeel ® PA-3 (Sodium Phytate; Aqua; Alcohol) |
| A1 | 0.30 wt.-% Xanthan Gum FEDCS-PC (Xanthan Gum) |
| B | 1.00 wt.-% TEGO ® Feel C 10 (Cellulose) |
| | 6.00 wt-% symbio@muls GC MB (Glyceryl Stearate Citrate; Cetearyl Alcohol; Glyceryl |
| | Caprylate) |
| | 6.00 wt.-% TEGOSOFT ® DC MB (Decyl Cocoate) |
| | 2.00 wt.-% dermosoft ® decalact sebum MB (Polyglyceryl-10 Laurate; Sodium |
| | Caproyl/Lauroyl Lactylate; Glycerin, Serenoa Serrulata Fruit Extract; Juniperus Communis |
| | Fruit Extract) |
| | 0.10 wt.-% dermofeel ® Toco 70 non GMO (Tocopherol; Helianthus Annuus (Sunflower) |
| | Seed Oil) |
| | 0.05 wt.-% Ceramide NP 15 |
| | 0.05 wt.-% SPHINOX Defenda (Hydroxybutyroyl Phytosphingosine) |
| | 0.1% TEGO Sterol 7-DHC (7-Dehydrocholesterol) |
| C | 3.00 wt.-% dermosoft ® 1388 eco (Glycerin; Aqua; Sodium Levulinate; Sodium Anisate) |
| | 0.15 wt.-% Perfume |

### 20.5 Lip Mask for Moisturization

| | |
|---|---|
| A | 0.02 wt.-% HyaCare ® (Sodium Hyaluronate) |
| | 9.96 wt.-% Glycerin |
| | ad 100 wt.-% Water |
| | 0.02 wt.-% Rouge Covarine W 3799 (CI12490; Aqua; Glycerin) |
| | 0.02 wt.-% Rouge Covarine W 3799 (CI12490; Aqua; Glycerin) |
| B | 8.00 wt.-% TEGOSOFT ® DC MB (Decyl Cocoate) |
| | 2.00 wt.-% TEGOSOFT ® OER MB (Oleyl Erucate) |
| | 3.00 wt.-% TEGOSOFT ® MM MB (Myristyl Myristate) |
| | 7.00 wt.-% TEGOSOFT ® P (Isopropyl Palmitate) |
| | 3.00 wt.-% ISOLAN ® GPS (Polyglyceryl-4 Diisostearate/ |
| | Polyhydroxystearate/Sebacate) |
| | 6.00 wt.-% TEGOSOFT ® SH MB (Stearyl Heptanoate) |
| | 1.00 wt.-% Cera Alba |
| | 10.00 wt.-% Candelilla Wax (Candelilla Cera) |
| | 3.00 wt.-% Copernicia Cerifera (Carnauba) Wax) |
| | 3.50 wt.-% Butyrospermum Parkii (Shea) Butter) |
| | 33.49 wt.-% SAT-Y-338073 (Yellow Iron Oxide) |
| | 33.49 wt.-% Schercemol PTID Ester (Triisostearoyl Polyglyceryl-3 |
| | Dimer Dilinoleate) |
| | 0.10 wt.-% Ceramide NP 15 |
| | 2.00 wt.-% HyaCare ® Filler CL MB (Aqua; Ethyhexyl Stearate; |
| | Sodium Hyaluronate Crosspolymer; Polyglyceryl-4 Diisostearate/ |
| | Polyhydroxystearate/Sebacate; Sodium Isostearate) |

### 20.6. Sleep Mask for Treatment of Photoaged Skin

| | |
|---|---|
| A | 0.50 wt.-% ABIL ® Care XL 80 MB (Bis-PEG/PPG-20/5 PEG/PPG-20/5 Dimethicone; |
| | Methoxy PEG/PPG-25/4 Dimethicone; Caprylic/Capric Triglyceride) |
| | 1.50 wt.-% ABIL ® Wax 9840 (Cetyl Dimethicone) |
| | 4.00 wt.-% TEGOSOFT ® OS (Ethylhexyl Stearate) |
| | 4.00 wt.-% TEGOSOFT ® CT MB (Caprylic/Capric Triglyceride) |
| | 0.10 wt.-% Ceramide NP 15 |
| | 3.00 wt.-% TEGO ® CBD Revitalize (Amaranthus Caudatus Seed Extract (and) Cholesterol |
| | (and Cannabidiol (and) Glyceryl Caprylate) |
| B | ad 100 wt.-% Water |
| | 5.00 wt.-% Glycerin |
| | 0.05 wt.-% KELCOGEL CG-HA (Gellan Gum) |
| | 0.40 wt.-% Carbomer (Carbomer Homopolymer C) |
| C | 2.00 wt.-% TEGO ® Natural Betaine (Betaine) |
| | 0.50 wt.-% TEGO ® Turmerone (Curcuma longa (turmeric) root extract) |
| D | q.s. Sodium Hydroxide (10% in water) |
| E | 0.80 wt.-% Verstatil ® PC (Phenoxyethanol; Caprylyl Glycol) |

### 20.7 Anti-Cellulite Body Mask

| | |
|---|---|
| A | ad 100 wt.-% Water, deionized |
| | 4.50 wt.-% Glycerin 99.5%, Ph. Eur. (Glycerin) |
| | 0.10 wt.-% dermofeel ® PA-3 (Sodium Phytate; Aqua; Alcohol) |
| | 0.80 wt.-% Xanthan Gum FEDCS-PC (Xanthan Gum) |
| B | 5.00 wt.-% Phytosqualan, refined, Olive based (Squalane) |
| B1 | 8.00 wt.-% symbio@muls GC MB (Glyceryl Stearate Citrate; Cetearyl |
| | Alcohol; Glyceryl Caprylate) |
| | 2.00 wt.-% TEGIN ® M Pellets MB (Glyceryl Stearate) |
| | 2.00 wt.-% KahlWax 8104 (Cera Alba) |
| | 2.00 wt.-% KahlWax 6614 (Camellia Sinensis Leaf Extract) |
| | 2.00 wt.-% Sheabutter, refined, organic (Butyrospermum Parkii Butter) |
| | 5.00 wt.-% Almond Oil, refined (Prunus Amygdalus Dulcis Oil) |
| | 3.00 wt.-% Avocado Oil, refined (Persea Gratissima Oil) |
| | 2.00 wt.-% Apricot Kernel Oil, refined (Prunus Armeniaca Kernel Oil) |
| | 0.20 wt.-% dermofeel ® Toco 70 non GMO (Tocopherol; Helianthus Annuus (Sunflower) |
| | Seed Oil) |
| | 0.10 wt.-% Ceramide NP 15 |
| | 1.00 wt.-% dermofeel ® TocoSkin (Tocopherol; Helianthus Annuus (Sunflower) Seed Oil) |
| C | 0.50 wt.-% TEGO ® Turmerone (Curcuma longa (turmeric) root extract) |
| | 0.50 wt.-% Pumpkin seed (Kurbiskern) organic extractive P-00025487 (Helianthus Annuus |
| | Hybrid Oil; Cucurbita Pepo Seed Extract; Rosmarinus Officinalis Leaf Extract) |
| | 3.5 wt.-% dermosoft ® 1388 eco NaL (Glycerin, Aqua, Sodium Levulinate, p-Anisic Acid, |
| | Sodium Hydroxide) |
| D | 1.00 wt.-% SKINMIMICS ® MB (Ceramide NP; Ceramide NG; Ceramide EOS; Ceramide |
| | EOP; Ceramide AP; Caprooyl Phytosphingosine; Caprooyl Sphingosine; Behenic Acid; |
| | Cholesterol; Cetearath-25; Glycerin; Cetyl Alcohol; Phenoxyethanol; Ethylhexylglycerin; |
| | Tocopherol; Ascorbyl Palmitate; Lecithin; Glyceryl Stearate; Glyceryl Oleate; Citric Acid; |
| | Aqua) |

### 20.8 Skin Calming After Shave Serum

| | |
|---|---|
| A | ad 100 wt.-% Water, deionized |
| | 0.10 wt.-% dermofeel ® PA-3 (Sodium Phytate; Aqua; Alcohol) |
| | 3.00 wt.-% dermosoft ® OM (Methylpropanediol; Caprylyl Glycol) |
| | 5.00 wt.-% Glycerin 99.5%, Ph. Eur. (Glycerin) |
| B | 2.50 wt.-% symbio@prot V MB (Hydrolyzed Vegetable Protein; Sodium Citrate; Magnesium |
| | Stearate; Xanthan Gum) |
| | 0.50 wt.-% TEGIN ® M Pellets MB (Glyceryl Stearate) |
| | 4.00 wt.-% TEGOSOFT ® DC MB (Decyl Cocoate) |
| | 0.50 wt.-% Phytosqualan (Squalane) |
| | 1.00 wt.-% TEGO ® Feel C 10 (Cellulose) |
| | 0.20 wt.-% dermofeel ® Toco 70 non GMO (Tocopherol; Helianthus Annuus (Sunflower) |
| | Seed Oil) |
| | 2.00 wt.-% dermosoft ® decalact sebum MB (Polyglyceryl-10 Laurate; Sodium |
| | Caproyl/Lauroyl Lactylate; Glycerin, Serenoa Serrulata Fruit Extract; Juniperus Communis |
| | Fruit Extract) |
| | 0.10 wt.-% Ceramide NP 15 |
| | 0.10 wt.-% Phytosphingosine (Phytosphingosine) |
| C | 3.00 wt.-% Ethanol denat. (Alcohol Denat.) |

### 20.9 Hair & Scalp Serum

| | |
|---|---|
| A | 2.00 wt.-% TEGOR Care LTP MB (Sorbitan Laurate; Polyglyceryl-4 Laurate; Dilauryl |
| | Citrate) |
| | 2.67 wt.-% TEGOSOFT ® CT MB (Caprylic/Capric Triglyceride) |
| | 1.33 wt.-% dermofeel ® sensolv MB (Isoamyl Laurate) |
| | 1.00 wt.-% Hairflux ® (Ceramide NG; Olea Europaea (Olive) Fruit Oil; Ricinus Communis |
| | (Castor) Seed Oil) |
| | 0.10 wt.-% Ceramide NP 15 |
| | 0.05% SPHINGONY ® (Sphinganine) |
| B | ad 100 wt.-% Water |
| | 3.00 wt.-% Glycerin |
| C | 2.00 wt.-% dermofeel ® sensolv MB (Isoamyl Laurate) |
| | 0.10 wt.-% Carbomer (Carbomer Homopolymer A) |
| | 0.10 wt.-% Acrylates/C10-C30 Alkyl Acrylate Crosspolymer (Carbomer Copolymer C) |
| D | 2.00 wt.-% Water |
| | 0.005 wt.-% TEGO ® Pep 3-Boost (Copper Tripeptide 34) |
| E | 1.00 wt.-% Verstatil ® PC (Phenoxyethanol; Caprylyl Glycol) |
| | q.s. Sodium Hydroxide (10% in water) |

### 20.10 Anti-Aging Low Viscous Face Serum

| | |
|---|---|
| A | 3.00 wt.-% TEGO ® Care PBS 6 MB (Polyglyceryl-6 Stearate (and) Polyglyceryl-6 |
| | Behenate) |
| | 0.50 wt.-% TEGIN ® M Pellets MB (Glyceryl Stearate) |
| | 4.00 wt.-% TEGOSOFT ® DEC (Diethylhexyl Carbonate) |
| | 5.00 wt.-% TEGOSOFT ® XC MB (Phenoxyethyl Caprylate) |
| | 3.00 wt.-% dermofeel ® sensolv MB (Isoamyl Laurate) |
| | 3.00 wt.-% TEGOR SP 13-1 MB (Poly C10-30 Alkyl Acrylate) |
| | 0.10 wt.-% Ceramide NP 15 |
| | 0.05 wt.-% Phytsophingosine SLC (Salicyloyl Phytosphingosine) |
| B | ad 100 wt.-% Water |
| | 3.00 wt.-% Glycerin |
| | 0.20 wt.-% Carbomer (Carbomer Homopolymer A) |
| | 0.10 wt.-% HyaCare ® 50 (Hydrolyzed Hyaluronic Acid) |
| C | q.s. Sodium Hydroxide (10% in water) |
| D | 0.50 wt.-% BeautiFerm Healerine (Water (Aqua); Ganoderma Lucium Extract; Glycerin; |
| | Propanediol; Xanthan Gum; 1,2-Hexanediol; Caprylyl Glycol) |
| E | 1.00 wt.-% Verstatil ® PC (Phenoxyethanol; Caprylyl Glycol) |
| Z | q.s. Perfume |

### 20.11 Anti-Acne Serum

| | |
|---|---|
| A | 2.00 wt.-% TEGOSOFT ® AC MB (Isoamyl Cocoate) |
| | 2.00 wt.-% TEGOSOFT ® OER MB (Oleyl Erucate) |
| | 1.00 wt.-% Octyldodecanol |
| | 1.00 wt.-% TEGOSOFT ® APM (PPG-3 Myristyl Ether) |
| | 0.50 wt.-% TEGO ® Turmerone (Curcuma longa (turmeric) root extract) |
| | 0.05 wt.-% Ceramide NP 15 |
| | 0.07 wt.-% SPHINGONY ® (Sphinganine) |
| B | 0.50 wt.-% TEGO ® Carbomer 341 ER (Acrylates/C10-30 Alkyl Acrylate Crosspolymer) |
| | 5.00 wt.-% Glycerin |
| | 3.00 wt.-% Alcohol |
| | ad 100 wt.-% Water |
| C | q.s. Sodium Hydroxide (10% in water) |
| Z | q.s. Preservative |

### 20.12 O/W After Shave Cream for Treatment of Irritated Skin

| | |
|---|---|
| A | 1.5 wt.-% AXOL ® C 62 Pellets MB (Glyceryl Stearate Citrate) |
| | 2.0 wt.-% TEGIN ® M Pellets MB (Glyceryl Stearate) |
| | 3.0 wt.-% TEGO ® Alkanol 1618 MB (Cetearyl Alcohol) |
| | 3.5 wt.-% TEGOSOFT ® TN (C12-15 Alkyl Benzoate) |
| | 2.0 wt.-% TEGOSOFT ® CR MB (Cetyl Ricinoleate) |
| | 1.0 wt.-% Triisostearin |
| | 0.5 wt.-% TEGOSOFT ® MM MB (Myristyl Myristate) |
| | 4.0 wt.-% Cyclopentasiloxane |
| | 2.0 wt.-% Macadamia Ternifolia Seed Oil |
| | 0.5 wt.-% Tocopheryl Acetate |
| B | 0.10 wt.-% Ceramide NP 15 |
| | 0.10 wt.-% Bodyflux ® Olive (Ceramide NP) |
| | 4.0 wt.-% Glycerin |
| | 0.5 wt.-% Panthenol |
| | 0.2 wt.-% Allantoin |
| | ad 100 wt.-% Water |
| C | 0.3 wt.-% Carbomer (Carbomer Homopolymer B) |
| | 1.2 wt.-% TEGOSOFT ® TN (C12-15 Alkyl Benzoate) |
| D | 0.5 wt.-% Bisabolol |
| E | q.s. Sodium Hydroxide (10% in water) |
| F | 7.0 wt.-% Alcohol |
| G | 0.7 wt.-% Euxyl PE 9010 (Phenoxyethanol, Ethylhexylglycerin) |
| Z | q.s. Perfume |

### 20.13 After-Sun Gel for Photoprotection

| | |
|---|---|
| A | 1.5 wt.-% TEGOSOFT ® liquid (Cetearyl Ethylhexanoate ) |
| | 0.6 wt.-% ABIL ® B 8832 (Bis-PEG/PPG-20/20 Dimethicone) |
| | 0.10 wt.-% Ceramide NP 15 |
| B | 0.3 wt.-% dermosoft ® GMCY MB (Glyceryl Caprylate) |
| | 0.5 wt.-% TEGOR Cosmo C 250 (1-Methylhydantoin-2-Imide) |
| | 0.03 wt.-% HyaCare ® (Sodium Hyaluronate) |
| | 3.00 wt.-% Glycerin |
| | 3.00 wt.-% Butylene Glycol |
| | ad 100 wt.-% Water, demineralized |
| | 0.2 wt.-% Carbomer (Carbomer Homopolymer C) |
| | 0.7 wt.-% Aristoflex ® HMB (Ammonium Acryloyldimethyltaurate, Behnenth-25 |
| | Methacrylate Crosspolymer) |
| C | 1.00 wt.-% dermosoft ® Hexiol (1,2-Hexanediol) |
| | q.s. Sodium Hydroxide |
| | 0.25 wt.-% Red 28 (0.02% in water) |
| D | ROVISOME ® Sensitive NG (Water/Aqua; Glycerin; Lecithin; Dipotassium Glycyrrhizate; |
| | Calendula Officinalis Extract; Lonicera Caprifolium Flower Extract; Lonicera Japonica |
| | Flower Extract; Benzyl Alcohol; Benzoic Acid; Dehydroacetic Acid) |

### 20.14 Sun Cream SPF 15

| | |
|---|---|
| A | 3.00 wt.-% TEGO ® Care PBS 6 MB (Polyglyceryl-6 Stearate (and) Polyglyceryl-6 |
| | Behenate) |
| | 8.00 wt.-% TEGOSOFT ® XC MB (Phenoxyethyl Caprylate) |
| | 5.00 wt.-% TEGOSOFT ® DEC (Diethylhexyl Carbonate) |
| | 3.00 wt.-% TEGIN ® M Pellets MB (Glyceryl Stearate) |
| | 2.00 wt.-% TEGO ® Alkanol 1618 MB (Cetearyl Alcohol) |
| | 1.00 wt.-% TEGOSOFT ® MM MB (Myristyl Myristate) |
| | 0.50 wt.-% ABIL ® 350 (Dimethicone) |
| | 0.50 wt.-% TEGO ® Feel C 10 (Cellulose) |
| | 2.50 wt.-% Diethylamino Hydroxybenzoyl Hexyl Benzoate |
| | 6.00 wt.-% Homosalate |
| | 0.50 wt.-% Tocopheryl Acetate |
| | 0.10 wt.-% Ceramide NP 15 |
| B | 11.58 wt.-% Water |
| | 0.42 wt.-% Sodium Hydroxide |
| | 3.00 wt.-% Eusolex 232 (Phenylbenzimidazole Sulfonic Acid) |
| C | ad 100 wt.-% Water |
| | 3.00 wt.-% Glycerin |
| | 1.00 wt.-% TEGO ® Pep 4-17 MB (Tetrapeptide-21; Glycerin; Butylene Glycol; Aqua) |
| | 1.00 wt.-% TEGO ® Pep 4-Even MB (Tetrapeptide-30; Glycerin; Water) |
| | 0.50 wt.-% TEGO ® Cosmo C 100 (Creatine) |
| D | 0.70 wt.-% Keltrol CG-SFT (Xanthan Gum) |
| E | 1.10 wt.-% Verstatil ® PC (Phenoxyethanol; Caprylyl Glycol) |
| | 0.50 wt.-% Perfume |

### 20.15 UV-Protection Hand Cream SPF 20

| | |
|---|---|
| A | 1.50 wt.-% TEGO ® Care PBS 6 MB (Polyglyceryl-6 Stearate (and) Polyglyceryl-6 |
| | Behenate) |
| | 2.50 wt.-% VARISOFT ® TA 100 (Distearyldimonium Chloride) |
| | 2.00 wt.-% Stearyl Alcohol |
| | 2.00 wt.-% TEGIN ® M Pellets MB (Glyceryl Stearate) |
| | 1.00 wt.-% TEGOSOFT ® SH MB (Stearyl Heptanoate) |
| | 5.00 wt.-% TEGOSOFT ® TN (C12-15 Alkyl Benzoate) |
| | 3.00 wt.-% Diethylamino Hydroxybenzoyl Hexyl Benzoate |
| | 3.00 wt.-% Ethylhexyl Salicylate |
| | 5.00 wt.-% Ethylhexyl Methoxycinnamate |
| | 0.50 wt.-% Tocopheryl Acetate |
| | 0.10 wt.-% Ceramide NP 15 |
| | 0.05% TEGO ® Sterol KCS (Potassium Cholesteryl Sulfate) |
| B | ad 100 wt.-% Water |
| | 3.00 wt.-% Glycerin |
| C | 1.50 wt.-% Nylon-12 |
| D | 2.00 wt.-% TEGO ® Natural Betaine (Betaine) |
| | 2.00 wt.-% LACTIL ® (Sodium Lactate; Sodium PCA; Glycine; Fructose; Urea; |
| | Nicacinamide; Inositol: Sodium Benzoate; Lactic Acid) |
| | 2.00 wt.-% Water |
| E | q.s. Preservative |
| | 0.50 wt.-% Perfume “Azura 231517” |

20.16 Sun Care Lotion SPF 30 with Insect Repellent Activity

| | |
|---|---|
| A | 3.50 wt.-% TEGOR Care PBS 6 MB (Polyglyceryl-6 Stearate (and) Polyglyceryl-6 |
| | Behenate) |
| | 2.00 wt.-% TEGOSOFT ® DC MB (Decyl Cocoate) |
| | 1.00 wt.-% TEGOSOFT ® TN (C12-15 Alkyl Benzoate) |
| | 0.50 wt.-% REWOPAL ® PIB 1000 (Polyisobutene) |
| | 0.50 wt.-% TEGIN ® M Pellets MB (Glyceryl Stearate) |
| | 0.50 wt.-% Stearyl Alcohol |
| | 15.00 wt.-% Insect Repellent 3535 (Ethyl Butylacetylaminopropionate) |
| | 3.00 wt.-% Tinosorb S (Bis-Ethylhexyloxyphenol Methoxyphenyl Triazine) |
| | 3.00 wt.-% Ethylhexyl Salicylate |
| | 6.00 wt.-% Homosalate |
| | 8.00 wt.-% Octocrylene |
| | 2.00 wt.-% Butyl Methoxydibenzoylmethane |
| | 2.00 wt.-% Ethylhexyl Methoxycinnamate |
| | 0.10 wt.-% Ceramide NP 15 |
| B | ad 100 wt.-% Water |
| | 3.00 wt.-% Glycerin |
| C | 0.60 wt.-% Sepigel 305 (Polyacrylamide (and) C13-14 Isoparaffin (and) Laureth-7) |
| D | 0.70 wt.-% EUXYL PE 9010 (Phenoxyethanol; Ethylhexylglycerin) |
| Z | q.s. Perfume |

### 20.17 O/W Foundation for Even Skin Tone

| | |
|---|---|
| A | 4.00 wt.-% TEGO ® Care PBS 6 MB (Polyglyceryl-6 Stearate (and) Polyglyceryl-6 |
| | Behenate) |
| | 1.50 wt.-% TEGO ® Alkanol 1618 MB (Cetearyl Alcohol) |
| | 1.00 wt.-% TEGIN ® M Pellets MB (Glyceryl Stearate) |
| | 1.00 wt.-% TEGOSOFT ® MM MB (Myristyl Myristate) |
| | 3.00 wt.-% TEGOSOFT ® TN (C12-15 Alkyl Benzoate) |
| | 2.00 wt.-% TEGOSOFT ® DEC (Diethylhexyl Carbonate) |
| | 2.00 wt.-% 9045 Silicone Elastomer Blend (Cyclopentasiloxane (and) Dimethicone |
| | Crosspolymer) |
| | 8.00 wt.-% Ethylhexyl Methoxycinnamate |
| | 5.00 wt.-% Ethylhexyl Salicylate |
| | 3.00 wt.-% Diethylamino Hydroxybenzoyl Hexyl Benzoate |
| | 3.00 wt.-% Bis-Ethylhexyloxyphenol Methoxyphenyl Triazine |
| | (Bis-Ethylhexyloxyphenol Methoxyphenyl Triazine) |
| | 0.10 wt.-% Ceramide NP 15 |
| B | ad 100 wt.-% Water, deionized |
| | 1.00 wt.-% Glycerin |
| | 0.15 wt.-% Xanthan Gum |
| C | 8.00 wt.-% Titanium Dioxide; CI 77891 |
| | 1.70 wt.-% Covarine Yellow WN 1792 GZ (CI 77492, Aqua, Glycerin, Xanthan Gum, |
| | Sodium Citrate) |
| | 0.40 wt.-% Covarine Red WN 3798 GZ (CI 77491, Aqua, Glycerin, Xanthan Gum, Sodium |
| | Citrate) |
| | 0.10 wt.-% Covarine Black WN 9798 GZ (CI 77499, Water, Glycerin, Xanthan Gum, |
| | Sodium Citrate) |
| | 2.00 wt.-% Glycerin |
| | 3.00 wt.-% Propylene Glycol |
| D | 2.00 wt.-% Nylon-12 |
| E- | 2.00 wt.-% ROVISOMER Q10 NG (Aqua; Glycerin; Lecithin; Ubiquinone; Tocopherol; |
| | Benzyl Alcohol; Benzoic Acid; Dehydroacetic Acid) |
| F | 0.70 wt.-% Euxyl PE 9010 (Phenoxyethanol, Ethylhexylglycerin) |

### 20.18 Face Care Mousse

| | |
|---|---|
| A | 2.50 wt.-% ABIL ® Care XL 80 MB (Bis-PEG/PPG-20/5 PEG/PPG-20/5 Dimethicone; |
| | Methoxy PEG/PPG-25/4 Dimethicone; Caprylic/Capric Triglyceride) |
| | 5.00 wt.-% Dimethicone (5 mPas) |
| | 4.00 wt.-% TEGOSOFT ® DEC (Diethylhexyl Carbonate) |
| | 3.00 wt.-% TEGOSOFT ® CT MB (Caprylic/Capric Triglyceride) |
| | 1.00 wt.-% TEGOSOFT ® CR MB (Cetyl Ricinoleate) |
| | 0.10 wt.-% Ceramide NP 15 |
| | 0.10 wt.-% SPHINOX ® Lift (Ceramide NP) |
| B | 4.00 wt.-% TEGO ® Pep UP (Tetrapeptide-4; Glycerin) |
| | 0.50 wt.-% TEGO ® Cosmo C 100 (Creatine) |
| | 0.50 wt.-% Panthenol |
| | 0.20 wt.-% Allantoin |
| | ad 100 wt.-% Water |
| C | 1.60 wt.-% TEGOSOFT ® OS (Ethylhexyl Stearate) |
| | 0.15 wt.-% Carbomer (Carbomer Homopolymer A) |
| | 0.15 wt.-% Carbomer (Carbomer Homopolymer C) |
| | 0.10 wt.-% Keltrol CG-SFT (Xanthan Gum) |
| D | q.s. Sodium Hydroxide (10% in water) |
| E | 1.00 wt.-% TEGO ® Betain 810 MB (Capryl/Capramidopropyl Betaine) |
| | 1.00 wt.-% REWOTERIC ® AM C MB (Sodium Cocoamphoacetate) |
| | 0.70 wt.-% Euxyl PE 9010 (Phenoxyethanol, Ethylhexylglycerin) |
| Z | q.s. Perfume |

### 20.19 Face Mask

| | |
|---|---|
| A | 1.00 wt.-% TEGOSOFT ® PC 41 MB (Polyglyceryl-4 Caprate) |
| | 1.30 wt.-% TEGOR Betain 810 MB (Capryl/Capramidopropyl Betaine) |
| | 1.00 wt.-% TEGO ® Solve 61 MB (Polyglyceryl-6 Caprylate; Polyglyceryl-3 Cocoate; |
| | Polyglyceryl-4 Caprate; Polyglyceryl-6 Ricinoleate) |
| | 1.00 wt.-% Glycerin |
| | 1.40 wt.-% Hexylene Glycol |
| | 0.10 wt.-% Ceramide NP 15 |
| B | 0.50 wt.-% TEGO ® Carbomer 140 (Carbomer) |
| | 2.00 wt.-% TEGO ® Natural Betaine (Betaine) |
| | ad 100 wt.-% Water |
| | 0.10 wt.-% Xanthan Gum |
| C | 2.50 wt.-% TEGO ® Feel C 10 (Cellulose) |
| | 0.50 wt.-% TEGO ® Turmerone (Curcuma longa (turmeric) root extract) |
| | 3.00 wt.-% Verstatil ® BOB (Benzyl Alcohol; Caprylyl Glycol; Benzoic Acid) |
| | q.s. wt.-% Sodium Hydroxide (10% in water) |
| D | ROVISOMER ACE NG (Glycerin; Aqua; Lecithin; Tocopheryl Acetate; 3-0-Ethyl Ascorbic |
| | Acid; Retinyl Palmitate; Tocopherol; Benzyl Alcohol; Benzoic Acid; Dehydroacetic Acid) |

### 20.20 After Shave Cream

| | |
|---|---|
| A | 2.00 wt.-% AXOL ® C 62 Pellets MB (Glyceryl Stearate Citrate) |
| | 2.00 wt.-% TEGIN ® M Pellets MB (Glyceryl Stearate) |
| | 3.00 wt.-% TEGO ® Alkanol 1618 MB (Cetearyl Alcohol) |
| | 4.00 wt.-% TEGOSOFT ® DEC (Diethylhexyl Carbonate) |
| | 3.50 wt.-% TEGOSOFT ® DC MB (Decyl Cocoate) |
| | 2.00 wt.-% TEGOSOFT ® CR MB (Cetyl Ricinoleate) |
| | 1.00 wt.-% TEGOSOFT ® OER MB (Oleyl Erucate) |
| | 0.50 wt.-% TEGOSOFT ® MM MB (Myristyl Myristate) |
| | 2.00 wt.-% Macadamia Ternifolia Seed Oil |
| | 0.50 wt.-% Tocopheryl Acetate |
| | 0.50 wt.-% Bisabolol |
| | 0.50 wt.-% dermosoft ® GMCY MB (Glyceryl Caprylate) |
| | 0.10 wt.-% Ceramide NP 15 |
| B | ad 100 wt.-% Water |
| | 4.0 wt.-% Glycerin |
| | 0.50 wt.-% Panthenol |
| | 0.20 wt.-% Allantoin |
| C | 0.40 wt.-% Keltrol CG-SFT (Xanthan Gum) |
| D | 7.00 wt.-% Alcohol |
| E | 0.01 wt.-% TEGO ® Natural Citrus (Citrus reticulata (tangerine) Peel Extract) |
| | 0.02 wt.-% TEGO ® Natural Matricaria (Chamomilla recutita (Matricaria) Flower Extract) |
| E | 1.50 wt.-% Verstatil ® SL non GMO (Aqua; Sodium Levulinate; Potassium Sorbate) |

### 20.21 Skin Tonic

| | |
|---|---|
| A | ad 100 wt.-% Water, deionized |
| | 4.00 wt.-% Glycerin 99.5%, Ph. Eur. (Glycerin) |
| | 0.20 wt.-% TEGO Natural Madecassoside (Madecassoside) |
| | 0.50 wt.-% Ajidew NL-50N (Sodium PCA; Aqua) |
| | 0.10 wt.-% dermofeel ® PA-3 (Sodium Phytate; Aqua; Alcohol) |
| B | 1.50 wt.-% symbio ®solv clear plus MB (Caprylyl/Capryl Glucoside; Aqua; Sodium Cocoyl |
| | Glutamate; Glyceryl Caprylate; Citric Acid; Polyglyceryl-6 Oleate; Sodium Surfactin) |
| | 1.00 wt.-% dermosoft ® decalact liquid MB (Sodium Caproyl/Lauroyl Lactylate; Triethyl Citrate) |
| | 0.10 wt.-% Ceramide NP 15 |
| | 25.00 wt.-% Ethanol denat. (Alcohol Denat.) |
| | 0.30 wt.-% Perfume |
| | 2.00 wt.-% dermofeel ® TEC eco (Triethyl Citrate) |

### 20.22 Hair and Body Shampoo

2.25 wt.-% RHEANCE One (Glycolipids)
0.50 wt.-% dermotecie sensoiv MB (Isoamyl Laurate)
9.70 wt.-% Lauryl Glucoside, 50%
20.00 wt.-% Coco-Glucoside (50%)
5.50 wt.-% Sodium Coco-Sulfato, 91%
0.10 wt.-% Ceramide NP 15
ad 100.00 wt.-% Water
6.80 wt.-% Glycerin
3.50 wt.-% Dermosoft® 1388 eco (Glycerin; Aqua; Sodium Levulinate; Sodium Anisate)
1.05 wt.-% TEGO® Remo 95 MB (Sorbitan Caprylate, Glyceryl Oleate) ad pH with Citric Acid

### 20.23 Micellar Water for Facial Cleansing

2.00 wt.-% TEGO® Solve 55 MB (Polyglyceryl-3 Caprylate/Caprate/Succinate; Propylene Glycol)
1.30 wt.-% TEGO® Betain 810 MB (Capryl/Capramidopropyl Betaine)
0.10 wt.-% Ceramide NP 15
0.05 wt.-% Perfume
ad 100.0 wt.-% Water
2.00 wt.-% TEGO® Natural Betaine (Betaine)
2.00 wt.-% TEGO Cosmo EPL (Polyepsilon Lysine Hydrochloride)
1.40 wt.-% Hexylene Glycol
1.00 wt.-% Glycerin
q.s. Preservative

### 20.24 Leave-In Hair Conditioner for Dry Hair

| | |
|---|---|
| A | 5.00 wt.-% TEGOR Sulfosuccinate DO 75 (Diethylhexyl Sodium Sulfosuccinate) |
| | 10.00 wt.-% dermofeel ® sensolv MB (Isoamyl Laurate) |
| | 48.00 wt.-% TEGOSOFT ® TN (C12-15 Alkyl Benzoate) |
| | 30.00 wt.-% Helianthus Annuus (Sunflower) Seed Oil) |
| | 0.10 wt.-% Ceramide NP 15 |
| B | 5.00 wt.-% TEGOSOFT ® PC 31 MB (Polyglyceryl-3 Caprate) |
| | 1.00 wt.-% VARISOFT ® EQ 100 (Bis-(Isostearoyl/Oleoyl Isopropyl) Dimonium |
| | Methosulfate) |

### 20.25 W/O Lotion

| | |
|---|---|
| A | 2.5 wt.-% ISOLAN ® GPS (Polyglyceryl-4 Diisostearate/Polyhydroxystearate/Sebacate) |
| | 10.0 wt.-% TEGOSOFT ® DEC (Diethylhexyl Carbonate) |
| | 9.0 wt.-% TEGOSOFT ® OP (Ethylhexyl Palmitate) |
| | 0.5 wt.-% Zinc Stearate |
| | 0.10 wt.-% Ceramide NP 15 |
| B | ad 100.0 wt.-% Water |
| | 3.0 wt.-% Glycerin |
| | 1.5 wt.-% Magnesium Sulfate Heptahydrate |
| Z | q.s. Preservative, Perfume |

### 20.26 Natural Hair Oil for Thin Hair

| | |
|---|---|
| A | 92.78 wt.-% dermofeel ® sensolv MB (Isoamyl Laurate) |
| | 1.00 wt.-% TEGOSOFT ® DC (Decyl Cocoate) |
| | 1.00 wt.-% Argania Spinosa Kernel Oil |
| | 1.00 wt.-% Brassica Oleracea Italica Seed Oil (Broccoli Seed Oil) |
| | 0.5% wt.-% Daucus Carota Sativa Juice; Sesamum Indicum Seed Oil (Carrot Oil |
| | 1.00 wt.-% Hairflux ® (Olea Europaea (Olive) Fruit Oil; Ricinus Communis (Castor Seed Oil; |
| | Ceramide NG) |
| | 1.00 wt.-% Coconut Oil |
| | 0.02 wt.-% Hippophae Rhamnoides Fruit Oil (Seabuckthorn Pulp Oil) |
| | 0.50 wt.-% dermosoft ® GMCY MB (Glyceryl Caprylate) |
| | 0.20 wt.-% dermofeel ® Toco 70 non GMO (Tocopherol; Helianthus Annuus (Sunflower) |
| | Seed Oil) |
| | 0.02 wt-% Ceramide NP 15 |
| | 0.10 wt.-% TEGO ® Natural Coleus (Coleus Forskohlii Extract) |
| B | 1.00 wt.-% Perfume |

### 20.27 Waterless Hand Bar—Solid Hand Cream for Improved Sustainability

| | |
|---|---|
| A | 45.80 wt.-% KahlJelly 7235 MB Natural (Ricinus Communis (Castor) Seed Oil; Beeswax; |
| | Copernicia Cerifera (Carnauba) Wax; Helianthus Annuus (Sunflower) Seed Oil; |
| | Tocopherol; Ascorbyl Palmitate) |
| | 20.00 wt.-% KahlComplex 6422 Veggisoft (Rhus Verniciflua Peel Wax; Simmondsia |
| | (Chinensis (Jojoba) Seed Oil; Cetearyl Alcohol; Myristyl Alcohol; Caprylic/Capric |
| | Triglyceride; Copernicia Cerifera (Carnauba) Wax; Helianthus Annuus (Sunflower) Seed Oil) |
| | 10.00 wt.-% Kahlwax 2225 Phyto (Helianthus Annuus (Sunflower Seed Wax; Olea |
| | Europaea (Olive) Oil; Rhus Verniciflua Peel Wax; Shorea Robusta Resin) |
| | 5.00 wt.-% Kahlwax 660/H Hydrolyzed Sunflower (Hydrolyzed Sunflower Seed Wax) |
| | 6.00 wt.-% TEGOSOFT ® OER MB (Oleyl Erucate) |
| | 5.00 wt.-% TEGOSOFT ® DC MB (Decyl Cocoate) |
| | 2.00 wt.-% TEGOSOFT ® CR MB (Cetyl Ricinoleate) |
| | 2.70 wt.-% Olea Europaea (Olive) Fruit Oil |
| | 2.00 wt.-% dermofeel ® viscolid MB (Hydrogenated Palm Oil; Hydrogenated Rapeseed Oil) |
| | 0.50 wt.-% dermosoft ® GMCY MB (Glyceryl Caprylate) |
| | 0.20 wt.-% dermofeel ® TocoSkin (Tocopherol; Helianthus Annuus (Sunflower) Seed Oil) |
| | 0.20 wt.-% dermofeel ® Toco 70 non GMO (Tocopherol; Helianthus Annuus (Sunflower |
| | Seed Oil) |
| | 0.10 wt.-% Ceramide NP 15 |
| | 0.50 wt.-% Perfume |

### 20.28 Shaping Body Butter

| | |
|---|---|
| A | 43.20 wt.-% Kahlwax 6237 Rapeseed (Hydrogenated Rapeseed Oil) |
| | 8.90 wt.-% Kahlwax 6607L (Helianthus Annuus Seed Cera; Tocopherol; Ascorbyl |
| | Palmitate) |
| | 3.00 wt.-% Kahlwax 2811 Rice (Oryza Sativa Bran Cera) |
| | 12.50 wt.-% Prunus Amygdalus Dulcis (Sweet Almond) Oil |
| | 11.50 wt.-% TEGOSOFT ® DC MB (Decyl Cocoate) |
| | 5.00 wt.-% TEGOSOFT ® OER MB (Oleyl Erucate) |
| | 3.00 wt.-% TEGOSOFT ® CR MB (Cetyl Ricinoleate) |
| | 2.00wt.-% TEGOSOFT ® MM MB (Myristyl Myristate) |
| | 2.00 wt.-% dermofeel ® viscolid MB (Hydrogenated Palm Oil; Hydrogenated Rapeseed Oil) |
| | 1.30 wt.-% Argania Spinosa (Argan) Kernel Oil |
| | 5.00 wt.-% Persea Gratissima (Avocado) Oil |
| | 0.50 wt.-% dermosoft ® GMCY MB (Glyceryl Caprylate) |
| | 0.20 wt.-% dermofeel ® TocoSkin (Tocopherol; Helianthus Annuus (Sunflower) Seed Oil) |
| | 0.20 wt.-% dermofeel ® Toco 70 non GMO (Tocopherol; Helianthus Annuus (Sunflower |
| | Seed Oil) |
| | 0.10 wt.-% Ceramide NP 15 |
| | 0.10 wt.-% TEGO ® Sterol KCS (Potassium Cholesteryl Sulfate) |
| | 1.00 wt.-% Covapate Uniwhite LC 9781 (Ricinus Communis (Castor) Seed Oil; CI 77891; |
| | Polyhydroxystearic Acid) |
| | 0.50 wt.-% Perfume |

### 20.29 Conditioning Shampoo Bar

| | |
|---|---|
| A | 45.90 wt.-% Pureact I-78, 80% (Sodium Cocoyl Isethionate, 80%) |
| | 2.50 wt.-% TEGO ® Feel C10 (Cellulose) |
| | 0.30 wt.-% Jaguar C-162 (Hydroxypropyl Guar (and) Hydroxypropyl Guar |
| | Hydroxypropyltrimonium Chloride) |
| | 30.40 wt.-% Oryza Sativa Starch (Resista Natural) |
| | 8.00 wt.-% TEGO ® Betain 810 MB (Capryl/Capramidopropyl Betaine) |
| | 1.80 wt.-% RHEANCER ONE (Glycolipids) |
| | 4.20 wt.-% SOPHANCE ® LA-A (Glycolipids; Sodium Lactate) |
| | 1.00 wt.-% TEGOSOFT ® OER MB (Oleyl Erucate) |
| | 3.00 wt.-% VARISOFT ® EQ 90 (Dioleoylethyl Hydroxyethylmonium Methosulfate) |
| | 2.00 wt.-% TEGO ® Remo 95 MB (Sorbitan Caprylate; Glyceryl Oleate) |
| | 0.50 wt.-% dermosoft ® GMCY (Glyceryl Caprylate) |
| | 0.10 wt.-% Ceramide NP 15 |
| | 0.30 wt-% Perfume |

### 20.30 Cleansing Foam for Sebaceous Skin

6.00 wt.-% RHEANCE® ONE (Glycolipids)
11.00 wt.-% TEGO® Betain 810 MB (Capryl/Capramidopropyl Betaine)
0.50 wt.-% TEGO® Remo 95 MB (Sorbitan Caprylate; Glyceryl Oleate)
2.00 wt.-% Dermosoft® decalact sebum MB (Polyglyceryl-10 Laurate; Sodium Caproyl/Lauroyl Lactylate; Glycerin; Serenoa serrulata Fruit Extract; Juniperus communis Fruit Extract)
4.00 wt.-% Dermosoft® Pentiol eco (Pentylene Glycol)
0.02 wt.-% Ceramide NP 15
76.40 wt.-% Water (Aqua)

### 20.31 AP/Deo Aerosol Spray

| | |
|---|---|
| A | 4.00 wt.-% ISOLAN ® 17 MB (Polyglyceryl-4 Diisostearate/Polyhydroxystearate/Sebacate; |
| | Caprylic/Capric Triglyceride; Polyglyceryl-3 Oleate; Diisostearoyl Polyglyceryl-3 Dimer |
| | Dilinoleate) |
| | 0.25 wt.-% Hydrogenated Castor Oil |
| | 0.25 wt.-% Microcrystalline Wax |
| | 20.50 wt.-% TEGOSOFT ® CT (Caprylic/Capric Triglyceride) |
| | 10.00 wt.-% TEGOSOFT ® DEC (Diethylhexyl Carbonate) |
| | 0.03 wt.-% Ceramide NP 15 |
| B | 31.97 wt.-% Water (Aqua) |
| | 2.00 wt.-% Glycerin |
| | 30.00 wt.-% Locron LIC (Aluminium Chlorohydrate (50% aq.)) |
| C | 1.00 wt.-% Perfume |

### 20.32 Odor-Reducing Deodorant Stick

| | |
|---|---|
| A | 2.50 wt.-% dermofeel NC MB (Polyglyceryl-3 Distearate; Glyceryl Stearate Citrate) |
| | 20.00 wt.-% TEGO ® Alkanol 1618 MB (Cetearyl Alcohol) |
| | 2.00 wt.-% TEGOSOFT ® MM (Myristyl Myristate) |
| | 10.00 wt.-% TEGOSOFT ® CR (Cetyl Ricinoleate) |
| | 1.00 wt.-% TEGODEOR PY 88 G (Zinc Ricinoleate) |
| | 10.00 wt.-% dermofeel ® viscolid MB (Hydrogenated Palm Oil; Hydrogenated Rapeseed Oil) |
| | 33.70 wt.-% Helianthus Annuus (Sunflower) Seed Oil |
| | 10.00 wt.-% TEGOSOFT ® OER MB (Oleyl Erucate) |
| | 0.10 wt.-% Ceramide NP 15 |
| | 4.00 wt.-% AEROSIL ® 200 (Silica) |
| | 6.00 wt.-% TEGO ® Feel C 10 (Cellulose) |
| | 0.30 wt.-% TEGO ® Cosmo P 813 MB (Polyglyceryl-3 Caprylate) |
| | 0.30 wt.-% dermosoft ® 250 eco (Phenylpropanol) |
| B | 0.10 wt.-% Bambusa Textilis Stem Extract (Bambusa Tetilis Stem Extract) |

### 20.33 Deo Wipe

| | |
|---|---|
| A | 4.00 wt.-% TEGODEO ® A30 eco (Propylene Glycol; Zinc Ricinoleate; Aqua; Arginine; Citric |
| | Acid) |
| | 7.00 wt.-% TEGO ® Solve 90 MB (Polyglyceryl-6 Caprylate; Polyglyceryl4 Caprate) |
| | 0.50 wt.-% TEGO ® Cosmo P 813 MB (Polyglyceryl-3 Caprylate) |
| | 0.25 wt.-% Texapon K12 G (Sodium Lauryl Sulfate) |
| | 0.05 wt.-% Ceramide NP 15 |
| | 1.20 wt.-% Verstatil ® TBG (Triethyl Citrate; Glyceryl Caprylate; Benzoic Acid) |
| | 0.30 wt.-% Perfume |
| | 2.00 wt.-% dermofeel ® tec eco (Triethyl Citrate) |
| | 0.30 wt.-% dermosoft ® decalact MB (Sodium Caproyl/Lauroyl Lactylate; Triethylcitrate; |
| | Salvia Officinalis (Sage) Oil) |
| B | 82.15 wt.-% Water (Aqua) |
| C | 2.25 wt.-% Citric Acid, 20% solution in water |

### 20.34 Cationic Body Butter

| | |
|---|---|
| A | 4.50 wt.-% VARISOFT ® TA 100 (Distearyldimonium Chloride) |
| | 4.00 wt.-% Stearyl Alcohol |
| | 3.00 wt.-% TEGIN ® M Pellets MB (Glyceryl Stearate) |
| | 5.00 wt.-% TEGOSOFT ® CT MB (Caprylic/Capric Triglyceride) |
| | 5.00 wt.-% TEGOSOFT ® AC MB (Isoamyl Cocoate) |
| | 5.00 wt.-% TEGOSOFT ® SH (Stearyl Heptanoate) |
| | 0.50 wt.-% ABIL ® 350 (Dimethicone) |
| | 0.10 wt.-% Ceramide NP 15 |
| B | 64.20 wt.-% Water (Aqua) |
| | 5.00 wt.-% Glycerin |
| C | 2.00 wt.-% ProContour (Aqua; Alcohol; Lecithin; Caffeine; Carnitine; Centella Asiatica Leaf |
| | Extract; Potassium Phosphate; Coleus Barbatus Root Extract; Tocopherol) |
| D | 1.00 wt.-% Verstatil PC (Phenoxyethanol; Caprylyl Glycol) |

### 20.35 Protecting Hand Cream

| | |
|---|---|
| A | 2.00 wt.-% TEGINACID ® C Pellets MB (Ceteareth-25 |
| | 3.50 wt.-% TEGIN ® M Pellets MB (Glyceryl Stearate) |
| | 2.00 wt.-% TEGO ® Alkanol 1618 MB (Cetearyl Alcohol) |
| | 7.50 wt.-% Isopropyl Myristate |
| | 2.00 wt.-% TEGOSOFT ® DC (Decyl Cocoate) |
| | 1.00 wt.-% ABIL ® 350 (Dimethicone) |
| | 2.00 wt.-% ABIL ® Wax 9840 (Cetyl Dimethicone) |
| | 1.50 wt.-% Cera Alba |
| | 0.08 wt.-% Ceramide NP |
| B | 3.00 wt.-% Glycerin |
| | 0.50 wt.-% Panthenol |
| | 71.90 wt.-% Water (Aqua) |
| C | 2.00 wt.-% TEGO enlight (Morus Alba Fruit Extract; Sodium Phytate; Aqua; Glycerin; |
| | Alcohol) |
| C | 1.00 wt.-% Euxyl PE 9010 (Phenoxyethanol; Ethylhexylglycerin) |

### 20.36 Refreshing Foot Cream

| | |
|---|---|
| A | 6.00 wt.-% TEGIN ® Pellets MB (Glyceryl Stearate SE) |
| | 2.00 wt.-% TEGO ® Alkanol 1618 MB (Cetearyl Alcohol) |
| | 1.00 wt.-% Stearic Acid |
| | 1.00 wt.-% ABIL ® 350 (Dimethicone) |
| | 5.00 wt.-% TEGOSOFT ® CT MB (Caprylic/Capric Triglyceride) |
| | 5.00 wt.-% TEGOSOFT ® OS (Ethylhexyl Stearate) |
| | 4.00 wt.-% Mineral Oil |
| | 1.00 wt.-% TEGO Natural Amaranth (Amaranthus Caudatus Seed Extract) |
| | 0.10 wt.-% Ceramide NP |
| B | 7.00 wt.-% Glycerin |
| | 64.40 wt.-% Water (Aqua) |
| | 3.00 wt.-% Sorbitol |
| C | 0.50 wt.-% SK-INFLUX ® V MB (Ceramide NP; Ceramide AP; Ceramide EOP; |
| | Phytosphingosine; Cholesterol; Sodium Lauroyl Lactylate; Carbomer; Xanthan Gum) |
| D | Verstatil ® PC (Phenoxyethanol; Caprylyl Glycol) |

### 20.37 Wrinkle-Reducing Eye Cream

| | |
|---|---|
| A | 3.00 wt.-% TEGO Care 450 MB (Polyglyceryl-3 Methylglucose Distearate) |
| | 1.50 wt.-% TEGIN M Pellets MB (Glyceryl Stearate) |
| | 1.50 wt.-% TEGO Alkanol 1618 MB (Cetearyl Alcohol) |
| | 2.00 wt.-% TEGOSOFT CT MB (Caprylic/Capric Triglyceride) |
| | 4.00 wt.-% TEGOSOFT OP MB(Ethylhexyl Palmitate) |
| | 9.50 wt.-% TEGOSOFT DC MB (Decyl Cocoate) |
| | 4.00 wt.-% Prunus Amygdalus Dulcis (Sweet Almond) Oil |
| | 0.10 wt.-% Ceramide NP 15 |
| | 0.35 wt.-% dermosoft GMCY MB (Glyceryl Caprylate) |
| B | 65.35 wt.-% Water (Aqua) |
| | 3.00 wt.-% Glycerin |
| C | 0.20 wt.-% Keltrol CG-SFT (Xanthan Gum) |
| D | 2.00 wt.-% TEGO Pep 4-17 MB (Tetrapeptide-21; Glycerin; Butylene Glycol; Water) |
| E | 2.00 wt % ROVISOME F.E.C. (Aqua; Alcohol; Lecithin; Escin; Ascophyllum Nodosum |
| | Extract; Caffeine; Potassium Phosphate; Tocopherol) |
| F | 1.50 wt.-% Verstatil SL non GMO (Aqua; Sodium Levulinate; Potassium Sorbate) |

1. A composition, comprising:
A) at least one self tanning agent,
B) at least one sphingolipid of a general formula I 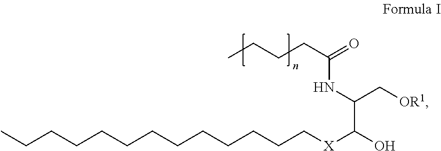
wherein
R¹ is H, phosphocholine, serine, ethanolamine or a sugar,
X is CH═CH, CH₂—CH₂ or CH₂—HCOH, and
n is selected from an integer from 3 to 8,
and/or
C) at least one sphingoid base selected from the group consisting of N-acetylphytosphingosine, phytosphingosine, sphingosine and sphinganine.

2. The composition according to claim 1, wherein the at least one self tanning agent is selected from the group consisting of dihydroxyacetone and erythrulose.

3. The composition according to claim 1, wherein
R¹ is H,
X is CH₂—HCOH, and
n is selected from an integer from 6 and 7
for the at least one sphingolipid of the general formula I.

4. The composition according to claim 1, wherein A) is comprised in an amount of 0.02% by weight to 1.50% by weight, percentages by weight being based on a total composition.

5. The composition according to claim 1, wherein B) is comprised in an amount of 0.5% by weight to 15.0% by weight, percentages by weight being based on a total composition.

6. A cosmetic formulation, comprising:
the composition according to claim 1.

7. The composition according to claim 6, further comprising:
at least one UV light protection filter substance.

8. A sphingolipid of a general formula Ib 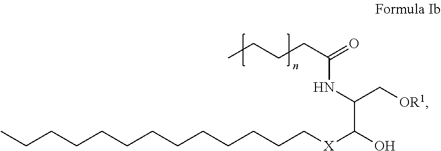
wherein
R¹ is H, phosphocholine, serine, ethanolamine or a sugar,
X is CH₂—HCOH, and
n 6,
or wherein
R¹ is H, phosphocholine, serine, ethanolamine or a sugar,
X is CH═CH or CH₂—HCOH, and
n 5.

9. A medicament, comprising:
the sphingolipid according to claim 8.

10. A method, comprising:
treating of acne, atopic dermatitis, psoriasis, herpes, warts, rosacea, skin cancer, pemphigus, alopecia, epidermolysis bullosa, scleroderma, ichthyosis, vitiligo and/or Raynaud's phenomenon, with the sphingolipid according to claim 8.

11. A composition, comprising:
the sphingolipid of the general formula Ib according to claim 8.

12. A method, comprising:
enhancing the self tanning effect of a self tanning agent by the at least one sphingolipid of the general formula I 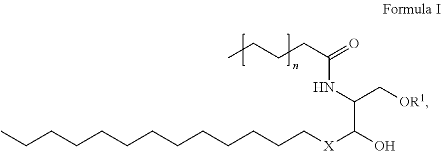
wherein
R¹ is H, phosphocholine, serine, ethanolamine or a sugar,
X is CH═CH, CH₂—CH₂ or CH₂—HCOH, and
n is an integer from 3 to 8,
and/or
by the at least one sphingoid base selected from the group consisting of N-acetylphytosphingosine, phytosphingosine, sphingosine and sphinganine.

13. A method, comprising:
tanning of a human skin by the composition according to claim 1.
